SEQUENCE LISTING (16 sequences)

SEQ ID NO: 1 - protein, 8 aa, mus musculus
GFSLTDYG

SEQ ID NO: 2 - protein, 7 aa, mus musculus
IWGDGTT

SEQ ID NO: 3 - protein, 10 aa, mus musculus
ARGRQFGFDY

SEQ ID NO: 4 - protein, 12 aa, mus musculus
QSLFNSRTRKNY

SEQ ID NO: 5 - protein, 3 aa, mus musculus
WAS

SEQ ID NO: 6 - protein, 8 aa, mus musculus
KQSYNLRT

SEQ ID NO: 7 - protein, 116 aa, mus musculus
QVQLKESGPGLVAPSQSLSITCTVSGFSLTDYGVYWVRQPPGKGLEWLGMIWGDGTTDYNSALKSRLSISKDNSKSQVFLKMNTLQTDDTARYYCARGRQFGFDYWGQGTTLTVSS

SEQ ID NO: 8 - protein, 112 aa, mus musculus
DIVMSQSPSSLAVSAGEKVTMRCKSSQSLFNSRTRKNYLAWYQQKPGQSPKLLIFWASIRESGVPDRFTGSGSGTDFTLTISSVQAEDLAVYYCKQSYNLRTFGGGTKLEIK

SEQ ID NO: 9 - DNA, 24 bp, mus musculus
gggttctcattaaccgactatggt

SEQ ID NO: 10 - DNA, 21 bp, mus musculus
atatggggtgatggaaccaca

SEQ ID NO: 11 - DNA, 30 bp, mus musculus
gccaggggtagacagttcgggtttgactac

SEQ ID NO: 12 - DNA, 36 bp, mus musculus
cagagtctgttcaacagtagaacccgaaagaactac

SEQ ID NO: 13 - DNA, 9 bp, mus musculus
tgggcatcc

SEQ ID NO: 14 - DNA, 24 bp, mus musculus
aagcaatcttataatcttcggacg

SEQ ID NO: 15 - DNA, 348 bp, mus musculus
caggtgcagctgaaggagtctgggcctggcctggtggcgccctcacagagcctgtccatcacatgcaccgtctcagggttctcattaaccgactatggtgtatactgggttcgccagcctccaggaaagggtctggagtggctgggaatgatatggggtgatggaaccacagactataattcagctctcaaatccagactgagcatcagtaaggacaactccaagagccaagttttcttaaaaatgaacactctgcaaactgatgacacagccaggtattactgtgccaggggtagacagttcgggtttgactactggggccaaggcaccacgctcacagtctcctca

SEQ ID NO: 16 - DNA, 336 bp, mus musculus
gacattgtgatgtcacagtctccatcctccctggctgtgtcagcaggagagaaggtcactatgaggtgcaaatccagtcagagtctgttcaacagtagaacccgaaagaactacttggcttggtaccaacagaaaccagggcagtctcctaaactgctgatcttctgggcatccattagggaatctggggtccctgatcgcttcacaggcagtggatctgggacagatttcactctcaccatcagcagtgtgcaggctgaagacctggcagtttattactgcaagcaatcttataatcttcggacgttcggtggaggcaccaagctggaaatcaaa

(19) United States
(12) Patent Application Publication
(10) Pub. No.: US 2014/0170677 A1
(43) Pub. Date: Jun. 19, 2014

(54) USE OF THE ANTIBODY I-3859 FOR THE DETECTION AND DIAGNOSIS OF CANCER

(71) Applicants: Christine Klinguer-Hamour, Groisy (FR); Alexandra Jouhanneaud, Bonneville (FR)

(72) Inventors: Christine Klinguer-Hamour, Groisy (FR); Alexandra Jouhanneaud, Bonneville (FR)

(73) Assignee: PIERRE FABRE MEDICAMENT, Boulogne-Billancourt (FR)

(21) Appl. No.: 14/235,235

(22) PCT Filed: Jul. 30, 2012
(86) PCT No.: PCT/EP2012/064876

§ 371 Date: Jan. 27, 2014

Related U.S. Application Data
(60) Provisional application No. 61/513,345, filed on Jul. 29, 2011.

(30) Foreign Application Priority Data
Jul. 29, 2011 (EP) 11306000.8

(51) Int. Cl. G01N 33/574; C07K 16/28

(52) U.S. Cl. CPC G01N 33/574; C07K 16/2866
USPC 435/7.23; 530/389.6; 530/391.3

(57) ABSTRACT

The present invention relates to the use of a novel, isolated anti-CXCR4 antibody in the diagnosis of cancer. In particular, methods for diagnosing and/or prognosing an oncogenic disorder associated with CXCR4 expression, are disclosed.

The present invention relates to the field of prognosis and/or diagnosis and/or therapy monitoring of a proliferative disease in a patient. More particularly, the invention relates to an antibody capable of binding specifically to the CXCR4, as well as the use of said antibody, and corresponding processes, for detecting and diagnosing pathological hyperproliferative oncogenic disorders associated with expression of CXCR4. In certain embodiments, the disorders are oncogenic disorders associated with increased expression of CXCR4 relative to normal or any other pathology connected with the overexpression of CXCR4. The invention finally comprises products and/or compositions or kits comprising at least such antibody for the prognosis and/or diagnostic and/or therapy monitoring of certain cancers.

Chemokines are small, secreted peptides that control the migration of leukocytes along a chemical gradient of ligand, known as chemokine gradient, especially during immune reactions (Zlotnick A. et al., 2000). They are divided into two major subfamilies, CC and CXC, based on the position of their NH₂-terminal cysteine residues, and bind to G protein coupled receptors, whose two major sub families are designated CCR and CXCR. More than 50 human chemokines and 18 chemokine receptors have been discovered so far.

Many cancers have a complex chemokine network that influences the immune-cell infiltration of tumor, as well as tumor cell growth, survival, migration and angiogenesis. Immune cells, endothelial cells and tumor cells themselves express chemokine receptors and can respond to chemokine gradients. Studies of human cancer biopsy samples and mouse cancer models show that cancer cell chemokine-receptor expression is associated with increase metastatic capacity. Malignant cells from different cancer types have different profiles of chemokine-receptor expression, but Chemokine receptor 4 (CXCR4) is most commonly found. Cells from at least 23 different types of human cancers of epithelial, mesenchymal and haematopoietic origin express CXCR4 receptor (Balkwill F. et al., 2004).

Chemokine receptor 4 (also known as fusin, CD184, LESTR or HUMSTR) exists as two isoforms comprising 352 or 360 amino acids. Isoform a has the amino acid sequence depicted under the Genbank accession number NP_{—}001008540, while isoform b has the amino acid sequence depicted under the Genbank accession number NP_{—}003458. Residue Asn11 is glycosylated, residue Tyr21 is modified by the addition of a sulfate group and Cys 109 and 186 are bond with a disulfide bridge on the extracellular part of the receptor (Juarez J. et al., 2004).

This receptor is expressed by different kind of normal tissues, naïve, non-memory T-cells, regulatory T cells, B-cells, neutrophils, endothelial cells, primary monocytes, dendritic cells, Natural Killer cells, CD34+ hematopoietic stem cells and at a low level in heart, colon, liver, kidneys and brain. CXCR4 plays a key role in leukocytes trafficking, B cell lymphopoiesis and myelopoiesis.

CXCR4 receptor is over-expressed in a large number of cancers including but not limited to lymphoma, leukemia, multiple myeloma, colon (Ottaiano A. et al., 2004), breast (Kato M. et al., 2003), prostate (Sun Y. X. et al., 2003), lungs [small-cell- and non-small-cell-carcinoma (Phillips R. J. et al., 2003)], ovary (Scotton C. J. et al., 2002), pancreas (Koshiba T. et al., 2000), kidneys, brain (Barbero S et al., 2002), glioblastoma and lymphomas.

The unique ligand of CXCR4 receptor described so far is the Stromal-cell-Derived Factor-1 (SDF-1) or CXCL12. SDF-1 is secreted in large amount in lymph nodes, bone marrow, liver, lungs and to a less extent by kidneys, brain and skin. CXCR4 is also recognized by an antagonistic chemokine, the viral macrophage inflammatory protein II (vMIP-II) encoded by human herpesvirus type III.

CXCR4/SDF-1 axis plays a key role in cancer and is implicated directly in migration, invasion leading to metastases. Indeed, cancer cells express CXCR4 receptor, they migrate and enter the systemic circulation. Then cancer cells are arrested in vascular beds in organs that produce high levels of SDF-1 where they proliferate, induce angiogenesis and form metastatic tumors (Murphy P M., 2001). This axis is also involved in cell proliferation via activation of Extracellular-signal-Regulated Kinase (ERK) pathway (Barbero S. et al., 2003) and angiogenesis (Romagnani P., 2004). Indeed, CXCR4 receptor and its ligand SDF-1 clearly promote angiogenesis by stimulating VEGF-A expression which in turns increases expression of CXCR4/SDF-1 (Bachelder R. E. et al., 2002). It is also known that tumor associated macrophages (TAM) accumulated in hypoxic areas of tumors and are stimulated to co-operate with tumor cells and promote angiogenesis. It was observed that hypoxia up-regulated selectively expression of CXCR4 in various cell types including TAM (Mantovani A. et al., 2004). It has been recently demonstrated that CXCR4/SDF-1 axis regulates the trafficking/homing of CXCR4+ hematopoietic stem/progenitor cells (HSC) and could play a role in neovascularization. Evidence indicates that besides HSC, functional CXCR4 is also expressed on stem cells from other tissues (tissue-committed stem cells=TCSCs) so SDF-1 may play a pivotal role in chemottracting CXCR4+ TCSCs necessary for organ/tissue regeneration but these TCSC may also be a cellular origin of cancer development (cancer stem cells theory). A stem cell origin of cancer was demonstrated for human leukemia and recently for several solid tumors such as brain and breast. There are several examples of CXCR4+ tumors that may derive from the normal CXCR4+ tissue/organ-specific stem cells such as leukemias, brain tumors, small cell lung cancer, breast cancer, hepatoblastoma, ovarian and cervical cancers (Kucia M. et al., 2005).

Targeting cancer metastases by interfering with CXCR4 receptor was demonstrated in vivo using a monoclonal antibody directed against CXCR4 receptor (Muller A. et al., 2001). Briefly, it was shown that a monoclonal antibody directed against CXCR4 receptor (Mab 173 R&D Systems) decreased significantly the number of lymph node metastases in an orthotopic breast cancer model (MDA-MB231) in SCID mice. Another study (Phillips R. J et al., 2003) also showed the critical role of SDF-1/CXCR4 axis in metastases in an orthotopic lung carcinoma model (A549) using polyclonal antibodies against SDF-1 but in this study there was no effect neither on tumor growth nor on angiogenesis. Several other studies described also the inhibition of either metastasis in vivo using siRNAs duplexes of CXCR4 (Liang Z. et al., 2005) biostable CXCR4 peptide antagonists (Tamamura H. et al., 2003) or tumor growth in vivo using small molecule antagonist of CXCR4 like AMD 3100 (Rubin J. B. et al., 2003; De Falco V. et al., 2007) or Mab (patent WO2004/059285 A2). Thus, CXCR4 is a validated therapeutic target for cancers.

Chemokine receptor 2 (CXCR2), another chemokine receptor is also described as an interesting target in oncology. Indeed, CXCR2 transmits an autocrine cell growth signal in several tumor cell types and can also affect tumor growth indirectly by promoting angiogenesis (Tanaka T. et al. 2005).

CXCR2 chemokine receptor encompasses 360 amino acids. It is expressed mainly in endothelial cells and especially during neovascularization. Several chemokines bind CXCR2 receptor: CXCL5, −6, −7, IL-8, GRO-α, -β and γ □ which belong to ERL+ pro-angiogenic chemokines. The CXCR2 receptor share sequence homologies with CXCR4 receptor: 37% sequence identity and 48% sequence homology. The CXCR2/ligands axis is involved in several tumor growth mechanisms such as metastasis (Singh R K. et al., 1994) cell proliferation (Owen J. D. et al., 1997) and in ERL+ chemokines-mediated angiogenesis (Strieter R. M. et al., 2004). Finally, tumor-associated macrophages and neutrophils are key elements of inflammatory-induced tumor growth and chemokines such as CXCL5, IL-8 and GRO-α initiate neutrophils recruitment.

Dimerization has emerged as a common mechanism for regulating the function of G-protein-coupled receptors, among these are chemokine receptors (Wang J. and Norcross M., 2008). Homo- and heterodimerization in response to chemokine binding has been shown to be required for the initiation and the alteration of signaling by a number of chemokine receptors. Growing evidence supports the concept that receptor dimers or oligomers are probably the basic functional unit of chemokine receptors. Chemokine receptor dimers are found in the absence of ligands and chemokines induce conformational changes of receptor dimers. CXCR4 is known to form homodimers but also heterodimers, for examples with the δ-opioid receptor (DOR) (Hereld D., 2008) or CCR2 (Percherancier Y. et al., 2005). In the latter example, peptides derived from the transmembrane domains of CXCR4 inhibited activation by blocking the ligand-induced conformational transitions of the dimer (Percherancier Y. et al., 2005). Another study showed that CXCR4-TM4 peptide, a synthetic peptide of the transmembrane region of CXCR4, decreases energy transfer between protomers of CXCR4 homodimers and inhibits SDF-1-induced migration and actin polymerization in malignant cells (Wang J. et al., 2006). More recently, it was also described that CXCR7 formed functional heterodimers with CXCR4 and enhanced SDF-1-induced signaling (Sierro F. et al., 2007). Other examples of constitutive heterodimers include studies showing CXCR1 and CXCR2 interact as well as forming respective homodimers. No interactions were noted for either of them with another GPCR (alpha(1A)-adrenoreceptor), indicating the specificity of CXCR1 and CXCR2 interaction (Wilson S. et al., 2005).

As previously mentioned, CXCR4 and CXCR2 receptors are interesting tumor targets. Interfering with those receptors should inhibit tumor growth and metastases in a very efficient way, by decreasing tumor cell proliferation, angiogenesis, tumor cell migration and invasion, neutrophils and macrophages recruitment by tumors and by inhibiting CXCR4 cancer stem cells.

Two monoclonal antibodies, referred as 515H7 and 414H5, which bind and induce conformational changes in both CXCR4 homodimers and CXCR4/CXCR2 heterodimers, and have strong anti-tumor activities, have been previously characterized (see WO 2010/037831). Moreover, the applicant has demonstrated the existence of such a CXCR4/CXCR2 heterodimer.

The present invention aims at providing at least one reagent, devoid of any in vivo activity in cancer models, which can be used as a diagnosis or prognosis tool for oncogenic disorders, especially those characterized by expression of CXCR4 or those that are mediated by aberrant CXCR4 expression.

The published patent application WO 2010/125162 discloses two anti-CXCR4 monoclonal antibodies, referred as 515H7 and 301aE5, and their uses in the field of the HIV treatment.

Surprisingly, the inventors have now demonstrated that the said antibody 301aE5 (also referred in the present specification as 301E5 or more preferably, by reference to the deposited hybridoma, I-3859: for the purpose of the present application, these terms are similar) does not have any in vivo activity in the field of the treatment of cancer, contrary to the other antibody 515H7 which presents strong anti-tumoral activities (as described in WO 2010/037831). In particular, I-3859 does not prevent the binding of the CXCR4 ligand to the receptor.

Moreover, the applicants found that the antibody I-3859 is capable of:
i) recognizing CXCR4 as monomers;
ii) recognizing CXCR4 as CXCR4/CXCR4 homodimers;
iii) recognizing CXCR4 as CXCR4/CXCR2 heterodimers;
iv) immunoprecipitating the CXCR4 from cellular lysat;
v) recognizing the CXCR4 at the surface of CXCR4-expressing cells by Fluorescence Activated Cell Sorting (FACS); and
vi) recognizing the CXCR4 by the immunohistochemistry method.

Because of these novel properties, which had never been disclosed previously for the antibody I-3859, the inventors have found that the said antibody can be used for identifying CXCR4-expressing cells and, in particular, CXCR4-expressing tumor cells.

Thus, the present invention relates to the use of said antibody for detecting the presence and/or location of CXCR4-expressing disease. The invention can then be utilized in diagnosing and/or prognosing, preferably in vitro, CXCR4-expressing diseases. Preferably, the said CXCR4-expressing disease is a cancer.

Another advantageous property of the antibody I-3859 of the invention is that it recognizes an epitope close to the epitope of the therapeutic monoclonal antibody 515H7. More particularly, as demonstrated in the experimental examples, I-3859 is capable of competing with the binding of the therapeutic antibody 515H7 to its epitope. The said I-3859 antibody can thus be used for, e.g., selecting patients to be treated with the 515H7 Mab. In particular, the antibody I-3859 of the invention could be used to check that the conformation of the CXCR4 present at the surface of the cells of a patient is similar to the conformation recognized by the antibody 515H7, indicating that the said patient is amenable to a 515H7 antibody-based therapy.

A first aspect of the invention relates to an isolated antibody, or an antigen-binding fragment or derivative thereof, that specifically binds to CXCR4 with high affinity, the said antibody being devoid of any in vivo activity. The said isolated antibody, or antigen-binding fragment or derivative thereof, can be used in methods for diagnosing or prognosing pathological hyperproliferative oncogenic disorders mediated by CXCR4 expression. In particular, the said isolated antibody can be used for in vivo imaging. Preferably, the isolated antibody of the invention binds human CXCR4.

In a preferred embodiment, an isolated antibody, or an antigen-binding fragment or derivative thereof, is provided for use in detecting the presence of a CXCR4-expressing tumor, wherein said antibody comprises at least one complementary determining region (CDR) chosen from CDRs comprising the amino acid sequence SEQ ID Nos. 1 to 6 or at least one CDR whose sequence has at least 80%, preferably 85%, 90%, 95% and 98% identity after optimal alignment with sequences 1 to 6.

More preferably, the invention comprises the antibodies, their antigen-binding fragments or derivatives, according to the present invention, obtained by genetic recombination or chemical synthesis.

According to a preferred embodiment, the antibody according to the invention, or its derived compounds or antigen-binding fragments, is characterized in that it consists of a monoclonal antibody.

A “monoclonal antibody”, as used herein, means an antibody arising from a nearly homogeneous antibody population. More particularly, the individual antibodies of a population are identical except for a few possible naturally-occurring mutations which can be found in minimal proportions. In other words, a monoclonal antibody consists of a homogeneous antibody arising from the growth of a single cell clone (for example a hybridoma, a eukaryotic host cell transfected with a DNA molecule coding for the homogeneous antibody, a prokaryotic host cell transfected with a DNA molecule coding for the homogeneous antibody, etc.) and is generally characterized by heavy chains of one and only one class and subclass, and light chains of only one type. Monoclonal antibodies are highly specific and are directed against a single antigen. In addition, in contrast with preparations of polyclonal antibodies which typically include various antibodies directed against various determinants, or epitopes, each monoclonal antibody is directed against a single epitope of the antigen.

A typical IgG antibody is composed of two identical heavy chains and two identical light chains that are joined by disulfide bonds. Each heavy and light chain contains a constant region and a variable region. Each variable region contains three segments called “complementarity-determining regions” (“CDRs”) or “hypervariable regions”, which are primarily responsible for binding an epitope of an antigen. They are usually referred to as CDR1, CDR2, and CDR3, numbered sequentially from the N-terminus. The more highly conserved portions of the variable regions are called the “framework regions”.

Three heavy chain CDRs and 3 light chain CDRs exist. The term CDR or CDRs is used here in order to indicate, according to the case, one of these regions or several, or even the whole, of these regions which contain the majority of the amino acid residues responsible for the binding by affinity of the antibody for the antigen or the epitope which it recognizes.

According to the invention, the CDRs of the antibody will be defined according to the IMGT numbering system. It will be obvious for the man skilled in the art to deduce the CDRs according to Kabat from the CDRs according to IMGT. The CDRs according to Kabat must be considered as part of the scope of the invention.

The IMGT unique numbering has been defined to compare the variable domains whatever the antigen receptor, the chain type, or the species [Lefranc M.-P., Immunology Today 18, 509 (1997)/Lefranc M.-P., The Immunologist, 7, 132-136 (1999)/Lefranc, M.-P., Pommié, C., Ruiz, M., Giudicelli, V., Foulquier, E., Truong, L., Thouvenin-Contet, V. and Lefranc, Dev. Comp. Immunol., 27, 55-77 (2003)]. In the IMGT unique numbering, the conserved amino acids always have the same position, for instance cystein 23 (1st-CYS), tryptophan 41 (CONSERVED-TRP), hydrophobic amino acid 89, cystein 104 (2nd-CYS), phenylalanine or tryptophan 118 (J-PHE or J-TRP). The IMGT unique numbering provides a standardized delimitation of the framework regions (FR1-IMGT: positions 1 to 26, FR2-IMGT: 39 to 55, FR3-IMGT: 66 to 104 and FR4-IMGT: 118 to 128) and of the complementarity determining regions: CDR1-IMGT: 27 to 38, CDR2-IMGT: 56 to 65 and CDR3-IMGT: 105 to 117. As gaps represent unoccupied positions, the CDR-IMGT lengths (shown between brackets and separated by dots, e.g. [8.8.13]) become crucial information. The IMGT unique numbering is used in 2D graphical representations, designated as IMGT Colliers de Perles [Ruiz, M. and Lefranc, M.-P., Immunogenetics, 53, 857-883 (2002)/Kaas, Q. and Lefranc, M.-P., Current Bioinformatics, 2, 21-30 (2007)], and in 3D structures in IMGT/3Dstructure-DB [Kaas, Q., Ruiz, M. and Lefranc, M.-P., T cell receptor and MHC structural data. Nucl. Acids. Res., 32, D208-D210 (2004)].

More particularly, according a first aspect, the invention relates to an antibody, or a antigen-binding fragment or derivative thereof, capable of binding specifically to the CXCR4, comprising i) a heavy chain comprising at least one of the following CDR-H1, CDR-H2 and CDR-H3, as defined according to IMGT numbering system, wherein CDR-H1 comprises the sequence SEQ ID No. 1, CDR-H2 comprises the sequence SEQ ID No. 2 and CDR-H3 comprises the sequence SEQ ID No. 3; and/or ii) a light chain comprising at least one of the following CDR-L1, CDR-L2 and CDR-L3, as defined according to IMGT numbering system, wherein CDR-L1 comprises the sequence SEQ ID No. 4, CDR-L2 comprises the sequence SEQ ID No. 5 and CDR-L3 comprises the sequence SEQ ID No. 6.

In still another embodiment, the invention can also be described as an antibody, or an antigen-binding fragment or derivative thereof, comprising:
a heavy chain comprising the following three CDRs as defined according to IMGT, respectively CDR-H1 having the sequence SEQ ID No. 1, CDR-H2 having the sequence SEQ ID No. 2 and CDR-H3 having the sequence SEQ ID No. 3, or a sequence having at least 80%, preferably 85%, 90%, 95% and 98% identity after optimal alignment with sequences SEQ ID Nos. 1, 2 or 3, respectively; and
a light chain comprising the following three CDRs as defined according to IMGT, respectively CDR-L1 having the sequence SEQ ID No. 4, CDR-L2 having the sequence SEQ ID No. 5 and CDR-L3 having the sequence SEQ ID No. 6, or a sequence having at least 80%, preferably 85%, 90%, 95% and 98% identity after optimal alignment with sequences SEQ ID Nos. 4, 5 or 6, respectively.

In the sense of the present invention, the “percentage identity” between two sequences of nucleic acids or amino acids means the percentage of identical nucleotides or amino acid residues between the two sequences to be compared, obtained after optimal alignment, this percentage being purely statistical and the differences between the two sequences being distributed randomly along their length. The comparison of two nucleic acid or amino acid sequences is traditionally carried out by comparing the sequences after having optimally aligned them, said comparison being able to be conducted by segment or by using an “alignment window”. Optimal alignment of the sequences for comparison can be carried out, in addition to comparison by hand, by means of the local homology algorithm of Smith and Waterman (1981) [Ad. App. Math. 2:482], by means of the local homology algorithm of Neddleman and Wunsch (1970) [J. Mol. Biol. 48:443], by means of the similarity search method of Pearson and Lipman (1988) [Proc. Natl. Acad. Sci. USA 85:2444] or by means of computer software using these algorithms (GAP, BESTFIT, FASTA and TFASTA in the Wisconsin Genetics Software Package, Genetics Computer Group, 575 Science Dr., Madison, Wis., or by the comparison software BLAST NR or BLAST P).

The percentage identity between two nucleic acid or amino acid sequences is determined by comparing the two optimally-aligned sequences in which the nucleic acid or amino acid sequence to compare can have additions or deletions compared to the reference sequence for optimal alignment between the two sequences. Percentage identity is calculated by determining the number of positions at which the amino acid or nucleotide residue is identical between the two sequences, dividing the number of identical positions by the total number of positions in the alignment window and multiplying the result by 100 to obtain the percentage identity between the two sequences.

For example, the BLAST program, “BLAST 2 sequences” (Tatusova et al., “Blast 2 sequences—a new tool for comparing protein and nucleotide sequences”, FEMS Microbiol., 1999, Lett. 174:247-250) available on the site http://www.ncbi.nlm.nih.gov/gorf/bl2.html, can be used with the default parameters (notably for the parameters “open gap penalty”: 5, and “extension gap penalty”: 2; the selected matrix being for example the “BLOSUM 62” matrix proposed by the program); the percentage identity between the two sequences to compare is calculated directly by the program.

For the amino acid sequence exhibiting at least 80%, preferably 85%, 90%, 95% and 98% identity with a reference amino acid sequence, preferred examples include those containing the reference sequence, certain modifications, notably a deletion, addition or substitution of at least one amino acid, truncation or extension. In the case of substitution of one or more consecutive or non-consecutive amino acids, substitutions are preferred in which the substituted amino acids are replaced by “equivalent” amino acids. Here, the expression “equivalent amino acids” is meant to indicate any amino acids likely to be substituted for one of the structural amino acids without however modifying the biological activities of the corresponding antibodies and of those specific examples defined below.

Equivalent amino acids can be determined either on their structural homology with the amino acids for which they are substituted or on the results of comparative tests of biological activity between the various antibodies likely to be generated.

As a non-limiting example, table 1 below summarizes the possible substitutions likely to be carried out without resulting in a significant modification of the biological activity of the corresponding modified antibody; inverse substitutions are naturally possible under the same conditions.

TABLE 1

| Original residue | Substitution(s) |
|---|---|
| Ala (A) | Val, Gly, Pro |
| Arg (R) | Lys, His |
| Asn (N) | Gln |
| Asp (D) | Glu |
| Cys (C) | Ser |
| Gln (Q) | Asn |
| Glu (E) | Asp |
| Gly (G) | Ala |
| His (H) | Arg |
| Ile (I) | Leu |
| Leu (L) | Ile, Val, Met |
| Lys (K) | Arg |
| Met (M) | Leu |
| Phe (F) | Tyr |
| Pro (P) | Ala |
| Ser (S) | Thr, Cys |
| Thr (T) | Ser |
| Trp (W) | Tyr |
| Tyr (Y) | Phe, Trp |
| Val (V) | Leu, Ala |

According to still another embodiment, the invention relates to the antibody I-3859, or one of its antigen-binding fragment or derivative, said antibody comprising a heavy-chain variable domain sequence comprising the amino acid sequence SEQ ID No. 7 or a sequence with at least 80%, preferably 85%, 90%, 95% and 98% identity after optimal alignment with sequence SEQ ID No. 7; and/or in that it comprises a light-chain variable domain sequence comprising the amino acid sequence SEQ ID No. 8, or a sequence with at least 80%, preferably 85%, 90%, 95% and 98% identity after optimal alignment with sequence SEQ ID No. 8.

In particular, the said antigen binding derivative consists of a binding protein comprising a peptide scaffold on which is grafted at least one CDR, the said CDR being grafted in such a way as to preserve all or part of the paratope recognition properties of the initial antibody. In a preferred embodiment, the said antigen binding protein is a fusion protein of a peptide scaffold and of the said at least one CDR.

One or more sequences among the six CDR sequences described in the present invention can also be present on the various immunoglobulin protein scaffolding. In this case, the protein sequence makes it possible to recreate a peptide skeleton suitable for the correct folding of the grafted CDRs, enabling them to preserve their paratope antigen-recognition properties.

The person skilled in the art will be aware of means to select the type of protein scaffold for CDR grafting. More particularly, it is known that to be selected, such scaffolds must meet as many criteria as possible (Skerra A., J. Mol. Recogn., 2000, 13:167-187):
good phylogenetic conservation;
known three-dimensional structure (as determined by, e.g., crystallography, NMR spectroscopy or any other technique known to a person skilled in the art);
small size;
few or no post-transcriptional modifications; and/or
easy to produce, express and purify.

The origin of such protein scaffolds can be, but is not limited to, the structures selected among: fibronectin and preferentially fibronectin type III domain 10, lipocalin, anticalin (Skerra A., J. Biotechnol., 2001, 74(4):257-75), protein Z arising from domain B of protein A of Staphylococcus aureus, thioredoxin A or proteins with a repeated motif such as the “ankyrin repeat” (Kohl et al., PNAS, 2003, vol. 100, No. 4, 1700-1705), the “armadillo repeat”, the “leucine-rich repeat” and the “tetratricopeptide repeat”. All such protein motifs have been extensively characterized in the art, and are thus well known to the skilled person.

As described above, such peptide scaffolds comprises from one to six CDRs arising from the original antibody. Preferably, but not being a requirement, a person skilled in the art will select at least one CDR from the heavy chain, the latter being known to be primarily responsible for the specificity of the antibody. The selection of one or more relevant CDRs is obvious to a person skilled in the art, who will then choose suitable known techniques (Bes et al., FEBS letters 508, 2001, 67-74).

By antigen-binding fragments of the antibody according to the invention, it must be understood, for example, the fragments Fv, scFv (sc=single chain), Fab, F(ab′)₂, Fab′, scFv-Fc or diabodies, or any fragment whose half-life has been increased by chemical modification, such as the addition of polyalkylene glycol such as polyethylene glycol (PEGylation) (PEGylated fragments are referred to as Fv-PEG, scFv-PEG, Fab-PEG, F(ab′)₂-PEG and Fab′-PEG), or by incorporation in a liposome, microspheres or PLGA, said fragments possessing at least one of the characteristic CDRs of the invention which is notably capable of exerting in a general manner activity, even partial, of the antibody from which it arises.

Preferably, said antigen-binding fragments will comprise or include a partial sequence of the variable heavy or light chain of the antibody from which they are derived, said partial sequence being sufficient to retain the same binding specificity as the antibody from which it arises and sufficient affinity, preferably at least equal to 1/100, more preferably at least 1/10 of that of the antibody from which it arises.

Such a antigen-binding fragment will contain at least five amino acids, preferably 6, 7, 8, 10, 15, 25, 50 or 100 consecutive amino acids of the sequence of the antibody from which it arises.

Preferably, these antigen-binding fragments will be of the types Fv, scFv, Fab, F(ab′)₂, F(ab′), scFv-Fc or diabodies, which generally have the same binding specificity as the antibody from which they result. According to the present invention, antigen-binding fragments of the antibody of the invention can be obtained from the antibodies described above by methods such as enzyme digestion, including pepsin or papain, and/or by cleavage of the disulfide bridges by chemical reduction. The antibody fragments can be also obtained by recombinant genetics techniques also known to a person skilled in the art or by peptide synthesis by means, for example, of automatic peptide synthesizers such as those sold by Applied BioSystems, etc.

The murine hybridoma capable of secreting the monoclonal antibody according to the invention, has been deposited at the CNCM, Institut Pasteur, Paris, France, on Oct. 22, 2007, under the number I-3859 Said hybridoma was obtained by the fusion of Balb/C immunized mice splenocytes and cells of the myeloma Sp 2/O—Ag 14 lines.

The monoclonal antibody, here referred to as 301aE5 or I-3859, or its antigen-binding fragment or derivative, is characterized in that it is secreted by said hybridoma.

The antibody I-3859 can also be described by its nucleic sequences, i.e. as comprising a heavy chain comprising a CDR-H1 encoded by the sequence SEQ ID No. 9, a CDR-H2 encoded by the sequence SEQ ID No. 10 and a CDR-H3 encoded by the sequence SEQ ID No. 11; and/or a light chain comprising a CDR-L1 encoded by the sequence SEQ ID No. 12, a CDR-L2 encoded by the sequence SEQ ID No. 13 and a CDR-L3 encoded by the sequence SEQ ID No. 14.

The antibody I-3859 comprises a heavy chain encoded by the nucleic sequences SEQ ID No. 15, or a nucleic sequence exhibiting a percentage identity of at least 80%, preferably 85%, 90%, 95% and 98%, after optimal alignment with SEQ ID No. 15; and/or a light chain encoded by the nucleic sequence SEQ ID No. 16, or a nucleic sequence exhibiting a percentage identity of at least 80%, preferably 85%, 90%, 95% and 98%, after optimal alignment with SEQ ID No. 16.

The terms “nucleic acid”, “nucleic sequence”, “nucleic acid sequence”, “polynucleotide”, “oligonucleotide”, “polynucleotide sequence” and “nucleotide sequence”, used interchangeably in the present description, mean a precise sequence of nucleotides, modified or not, defining a fragment or a region of a nucleic acid, containing unnatural nucleotides or not, and being either a double-strand DNA, a single-strand DNA or transcription products of said DNAs.

“Nucleic sequences exhibiting a percentage identity of at least 80%, preferably 85%, 90%, 95% and 98%, after optimal alignment with a preferred sequence” means nucleic sequences exhibiting, with respect to the reference nucleic sequence, certain modifications such as, in particular, a deletion, a truncation, an extension, a chimeric fusion and/or a substitution, notably punctual. Preferably, these are sequences which code for the same amino acid sequences as the reference sequence, this being related to the degeneration of the genetic code, or complementarity sequences that are likely to hybridize specifically with the reference sequences, preferably under highly stringent conditions, notably those defined below.

Hybridization under highly stringent conditions means that conditions related to temperature and ionic strength are selected in such a way that they allow hybridization to be maintained between two complementarity DNA fragments. On a purely illustrative basis, the highly stringent conditions of the hybridization step for the purpose of defining the polynucleotide fragments described above are advantageously as follows.

DNA-DNA or DNA-RNA hybridization is carried out in two steps: (1) prehybridization at 42° C. for three hours in phosphate buffer (20 mM, pH 7.5) containing 5×SSC (1×SSC corresponds to a solution of 0.15 M NaCl+0.015 M sodium citrate), 50% formamide, 7% sodium dodecyl sulfate (SDS), 10×Denhardt's, 5% dextran sulfate and 1% salmon sperm DNA; (2) primary hybridization for 20 hours at a temperature depending on the length of the probe (i.e.: 42° C. for a probe >100 nucleotides in length) followed by two 20-minute washings at 20° C. in 2×SSC+2% SDS, one 20-minute washing at 20° C. in 0.1×SSC+0.1% SDS. The last washing is carried out in 0.1×SSC+0.1% SDS for 30 minutes at 60° C. for a probe >100 nucleotides in length. The highly stringent hybridization conditions described above for a polynucleotide of defined size can be adapted by a person skilled in the art for longer or shorter oligonucleotides, according to the procedures described in Sambrook, et al. (Molecular cloning: a laboratory manual, Cold Spring Harbor Laboratory; 3rd edition, 2001).

In another aspect, the invention relates to an antibody of the invention, or an antigen-binding fragment or derivative thereof, for the in vitro or ex vivo diagnosis or prognosis of an oncogenic disorder associated with expression of CXCR4.

The invention thus relates to a method of in vitro or ex vivo diagnosing of prognosing an oncogenic disorder associated with expression of CXCR4, comprising the step of testing the binding of an antibody of the invention, or an antigen-binding fragment or derivative thereof, to CXCR4.

In particular, the invention provides the use of the antibody I-3859, or an antigen-binding fragment or derivative thereof, for in vitro diagnosis or prognosis of an oncogenic disorder associated with expression of CXCR4.

Importantly, the said antibody, or antigen-binding fragment or derivative thereof, does not have any in vivo anti-tumoral activity. This property is clearly advantageous for diagnosis application since it allows screening patient, or monitoring the progress of a treatment with an antibody which does not have any impact or consequence on the said patient. This property makes the antibody of the invention a preferred tool for screening patients to be treated as it will have no deleterious effect on the patient. The antibody of the invention, or a antigen-binding fragment or derivative thereof, will find use in various medical or research purposes, including the detection, diagnosis, and staging of various pathologies associated with expression of CXCR4.

“Diagnosing” a disease as used herein refers to the process of identifying or detecting the presence of a pathological hyperproliferative oncogenic disorder associated with or mediated by expression of CXCR4, monitoring the progression of the disease, and identifying or detecting cells or samples that are indicative of a disorder associated with the expression of CXCR4.

“Prognosis” as used herein means the likelihood of recovery from a disease or the prediction of the probable development or outcome of a disease. For example, if a sample from a subject is negative for staining with the antibody of the invention, then the “prognosis” for that subject is better than if the sample is positive for CXCR4 staining. Samples may be scored for CXCR4 expression levels on an appropriate scale as it will be more detailed hereinafter.

The antibody can be present in the form of an immunoconjugate or of a labeled antibody to obtain a detectable/quantifiable signal. When used with suitable labels or other appropriate detectable biomolecules or chemicals, the antibody of the invention is particularly useful for in vitro and in vivo diagnosis and prognosis applications.

Labels for use in immunoassays are generally known to those skilled in the art and include enzymes, radioisotopes, and fluorescent, luminescent and chromogenic substances, including colored particles such as colloidal gold or latex beads. Suitable immunoassays include enzyme-linked immunosorbent assays (ELISA). Various types of labels and methods of conjugating the labels to the antibodies of the invention are well known to those skilled in the art, such as the ones set forth below.

As used herein, the term “an oncogenic disorder associated with expression of CXCR4” is intended to include diseases and other disorders in which the presence of high levels of CXCR4 (aberrant) in a subject suffering from the disorder has been shown to be or is suspected of being either responsible for the pathophysiology of the disorder or a factor that contributes to a worsening of the disorder. Alternatively, such disorders may be evidenced, for example, by an increase in the levels of CXCR4 on the cell surface in the affected cells or tissues of a subject suffering from the disorder. The increase in CXCR4 levels may be detected using the antibody I-3859 of the invention.

In certain embodiments, “increased expression” as it relates to CXCR4 refers to protein or gene expression levels that demonstrate a statistically significant increase in expression (as measured by RNA expression or protein expression) relative to a control.

A preferred aspect of the invention is a method for detecting in vitro or ex vivo the presence of a CXCR4-expressing tumor in a subject, said method comprising the steps of:

(a) contacting a biological sample from the subject with the antibody of the invention, or an antigen-binding fragment or derivative thereof, and

(b) detecting the binding of said antibody with the said biological sample.

The binding of the antibody of the invention may be detected by various assays available to the skilled artisan. Although any suitable means for carrying out the assays are included within the invention, FACS, ELISA, western blotting and immunohistochemistry (IHC) can be mentioned in particular.

In another embodiment, the invention relates to a method for detecting in vitro or ex vivo the location of a CXCR4-expressing tumor in a subject, said method comprising the steps of:

(a) contacting a biological sample from the subject with the antibody of the invention, or an antigen-binding fragment or derivative thereof, and

(b) detecting the binding of the said antibody, or antigen-binding fragment or derivative thereof, with the sample.

As for the detection of the presence of an expressing tumor, many techniques known by the man skilled in the art can be used. Preferred methods include IHC and FACS.

The invention also relates to a method for detecting in vitro or ex vivo the percentage of cells expressing CXCR4 in a subject, said method comprising the steps of:

(a) contacting a biological sample from the subject with the antibody of the invention, or an antigen-binding fragment or derivative thereof, and

(b) quantifying the percentage of cells expressing CXCR4 in the said biological sample.

Another aspect of the invention relates to a method for determining in vitro or ex vivo the expression level of CXCR4 in a CXCR4-expressing tumor from a subject, said method comprising the steps of:

(a) contacting a biological sample from the subject with the antibody of the invention, or an antigen-binding fragment or derivative thereof, and

(b) quantifying the level of antibody binding to CXCR4 in said biological sample.

As will be apparent to the skilled artisan, the level of antibody binding to CXCR4 may be quantified by any means known to the person of skills in the art. Preferred methods involve the use of immunoenzymatic processes, such as ELISA assays, immunofluorescence, IHC, radio-immunoassay (RIA), or FACS.

Preferably, the biological sample is a biological fluid, such as serum, whole blood cells, a tissue sample or a biopsy of human origin. The sample may for example include, biopsied tissue, which can be conveniently assayed for the presence of a pathological hyperproliferative oncogenic disorder associated with expression of CXCR4.

Yet another aspect of the invention relates to a method for determining in vitro or ex vivo the expression level of CXCR4 in a tumor from a subject, the said method comprising the steps of:

(a) contacting a sample from the subject with an antibody according the invention, or an antigen binding fragment or derivative thereof, and

(b) quantifying the level of binding of the said antibody, or antigen binding fragment or derivative thereof, to CXCR4 in the sample.

Once a determination is made of the amount of CXCR4 present in the test sample, the results can be compared with those of control samples, which are obtained in a manner similar to the test samples but from individuals that do not have a hyperproliferative oncogenic disorder associated with expression of CXCR4. If the level of CXCR4 is significantly elevated in the test sample, it may be concluded that there is an increased likelihood of the subject from which it was derived has or will develop said disorder.

The invention relates, more particularly, to a process of in vitro or ex vivo diagnosis or prognosis of a CXCR4-expressing tumor, wherein said process comprises the steps of (i) determining the expression level of CXCR4 as above described, and (ii) comparing the expression level of step (i) with a reference expression level of CXCR4 from normal tissue or a non expressing CXCR4 tissue.

With regards to the development of targeted antitumor therapy, the diagnosis with immunohistological techniques gives in situ information on the receptor expression level and thus enables to select patients susceptible to be treated following the expression level of receptors needed for such treatment.

Stage determination has potential prognosis value and provides criteria for designing optimal therapy. Simpson et al., J. Clin. Oncology 18:2059 (2000). For example, treatment selection for solid tumors is based on tumor staging, which is usually performed using the Tumor/Node/Metastasis (TNM) test from the American Joint Committee on Cancer (AJCC). It is commonly acknowledged that, while this test and staging system provides some valuable information concerning the stage at which solid cancer has been diagnosed in the patient, it is imprecise and insufficient. In particular, it fails to identify the earliest stages of tumor progression.

The invention relates to a method for determining in vitro or ex vivo the scoring of a tumor of a subject, said method comprising the steps of:

(a) contacting a biological sample from the subject with an antibody, or an antigen-binding fragment or derivative thereof, capable of binding specifically to CXCR4;

(b) quantifying the level of binding of the said antibody, or antigen-binding fragment or derivative thereof, to CXCR4 in the said biological sample; and

(c) scoring the tumor by comparing the quantified level of binding of the said antibody, or antigen-binding fragment or derivative thereof, from the subject to an appropriate scale,

characterized in that the said antibody, or antigen-binding fragment or derivative thereof, comprises i) a heavy chain comprising the following three CDRs, respectively CDR-H1 having the sequence SEQ ID No. 1, CDR-H2 having the sequence SEQ ID No. 2 and CDR-H3 having the sequence SEQ ID No. 3; and ii) a light chain comprising the following three CDRs, respectively CDR-L1 having the sequence SEQ ID No. 4, CDR-L2 having the sequence SEQ ID No. 5 and CDR-L3 having the sequence SEQ ID No. 6.

In a preferred embodiment, the antibody for diagnosis is capable of binding the targeted receptor when tissue samples are, formalin fixed-, formol substituted fixed-, Glyco-fixx fixed-, paraffin embedded and/or frozen.

Any conventional hazard analysis method may be used to estimate the prognostic value of CXCR4. Representative analysis methods include Cox regression analysis, which is a semiparametric method for modeling survival or time-to-event data in the presence of censored cases (Hosmer and Lemeshow, 1999; Cox, 1972). In contrast to other survival analyses, e.g. Life Tables or Kaplan-Meyer, Cox allows the inclusion of predictor variables (covariates) in the models. Using a convention analysis method, e.g., Cox one may be able to test hypotheses regarding the correlation of CXCR4 expression status of in a primary tumor to time-to-onset of either disease relapse (disease-free survival time, or time to metastatic disease), or time to death from the disease (overall survival time). Cox regression analysis is also known as Cox proportional hazard analysis. This method is standard for testing the prognostic value of a tumor marker on patient survival time. When used in multivariate mode, the effect of several covariates are tested in parallel so that individual covariates that have independent prognostic value can be identified, i.e. the most useful markers. The term negative or positive “CXCR4 status” can also be referred as [CXCR4(−)] or [CXCR4(+)].

A sample may be “scored” during the diagnosis or monitoring of cancer. In its simplest form, scoring may be categorical negative or positive as judged by visual examination of samples by immunohistochemistry. More quantitative scoring involves judging the two parameters intensity of staining and the proportion of stained (“positive”) cells that are sampled.

“CXCR4 status” within the meaning of the invention, relates to the classification of tumor to a CXCR4 positive [CXCR4(+)] or CXCR4 negative [CXCR4(−)] class based on the determination of the expression level of the CXCR4 as measured by any methods such as immunohistochemistry (IHC), FACS, or other methods known by the man skilled in the art.

In an embodiment of the invention, to ensure standardization, samples may be scored for CXCR4 expression levels on different scales, most of them being based on an assessment of the intensity of the reaction product and the percentage of positive cells (Payne et al., Predictive markers in breast cancer—the present, Histopathology 2008, 52, 82-90).

In a more preferred embodiment of the process according to the invention, said scoring comprises using an appropriate scale based on two parameters which are the intensity of the staining and the percentage of positive cells.

As a first example, by analogy with the Quick Allred scoring for IHC assessment of oestrogen receptor and progesterone receptor, samples may be scored for CXCR4 expression levels on a global scale from 0 to 8 combining scores for intensity of reactivity and for the proportion of cells stained (Harvey J M, Clarck G M, Osborne C K, Allred D C; J. Clin. Oncol. 1999; 17; 1474-1481). More particularly, the first criteria of intensity of reactivity is scored on a scale from 0 to 3, 0 corresponding to “No reactivity” and 3 corresponding to “Strong reactivity”. The second criteria of proportion reactive is scored on a scale from 0 to 5, 0 corresponding to “No reactivity” and 5 to “67-100% proportion reactive”. The intensity of reactivity score and the proportion reactive score are then summed to produce total score of 0 through 8.

A total score of 0-2 is regarded as negative while a total score of 3-8 is regarded as positive.

According to this scale, the terms negative or positive “CXCR4 status” of tumors used in the present description refers to levels of expression of CXCR4 that correspond to scores 0-2 or 3-8 on the Allred scale, respectively.

Table 2 hereinafter illustrates the guidelines for interpreting IHC results according to Allred method.

TABLE 2

| Intensity of immunoreactivity | Score 1 | Proportion reactive | Score 2 |
|---|---|---|---|
| No reactivity | 0 | No reactivity | 0 |
| Weak reactivity | 1 | <1% | 1 |
| Moderate reactivity | 2 | 1-10% | 2 |
| Strong reactivity | 3 | 11-33% | 3 |
| | — | 34-66% | 4 |
| | — | 67-100% | 5 |

| Total Score (Score 1 + Score 2) | Interpretation |
|---|---|
| 0-2 | Negative |
| 3-8 | Positive |

In a preferred embodiment, the process according to the invention refers to an appropriate scale which is a scale of 0 to 8 wherein no reactivity is scored 0, and a strong reactivity in a proportion of 67-100% reactive is scored 8.

In another embodiment of the invention, it is described a process of determining in vitro or ex vivo the status of a tumor from a subject, wherein said process comprises the steps of (a) scoring a tumor from a subject according to the Allred scale; and (b) determining that the status of the tumor is [CXCR4(+)] with an Allred score of 3 to 8; or (c) determining that the status of the tumor is [CXCR4(−)] with an Allred score of 0 to 2.

In a particular aspect of the invention, a tumor is [CXCR4(+)] with an Allred score of 3.

In a particular aspect of the invention, a tumor is [CXCR4(+)] with an Allred score of 4.

In a particular aspect of the invention, a tumor is [CXCR4(+)] with an Allred score of 5.

In a particular aspect of the invention, a tumor is [CXCR4(+)] with an Allred score of 6.

In a particular aspect of the invention, a tumor is [CXCR4(+)] with an Allred score of 7.

In a particular aspect of the invention, a tumor is [CXCR4(+)] with an Allred score of 8.

In another particular aspect of the invention, a tumor is [CXCR4(+)] with an Allred score of 3 to 8.

As a second example, by analogy with the conventional scoring for IHC assessment of HER-2 receptor for example, samples may be scored for CXCR4 expression levels on a somewhat simpler scoring method integrating the intensity of staining (preferentially membranous staining) and the proportion of cells that display staining into a combined scale from 0 to 3+.

In this scale, referred as the simplified scale, 0 and 1+ are negative whereas 2+ and 3+ represents positive staining. Nevertheless, scores 1+-3+ can be recoded as positive because each positive score may be associated with significantly higher risk for relapse and fatal disease when compared to score 0 (negative), but increasing intensity among the positive scores may provide additional risk reduction.

Generally speaking, the terms negative or positive “CXCR4 status” of tumors used in the present description refers to levels of expression of CXCR4 that correspond to scores 0-1+ or 2+-3+ on the simplified scale, respectively. Only complete circumferential membranous reactivity of the invasive tumor should be considered and often resembled a “chicken wire” appearance. Under current guidelines, samples scored as borderline (score of 2+ or 3+) for CXCR4 are required to undergo further assessment. The IHC analysis should be rejected, and either repeated or tested by FISH or any other method if, as non limitative example, controls are not as expected, artifacts involve most of the sample and the sample has strong membranous positivity of normal breast ducts (internal controls) suggesting excessive antigen retrieval.

For more clarity, table 3 hereinafter summarizes these parameters.

TABLE 3

| CXCR4 | |
|---|---|
| status | IHC description |
| 0 | No reactivity or membranous reactivity in less than 10% of |
| | tumour cells. |
| 1+ | Faint/barely perceptible membranous reactivity is detected |
| | in more than 10% of tumour cells. The cells are |
| | immunoreactive only in part of the membrane. |
| 2+ | Weak to moderate complete membranous reactivity is seen |
| | in more than 10% of tumour cells. |
| 3+ | Strong complete reactivity is seen in more than 10% of tumour |
| | cells. |

In a preferred embodiment, the process according to the invention refers to an appropriate scale which is a scale of 0 to 3+ wherein no membranous reactivity of tumor cells is scored 0, and strong complete reactivity in more than 10% of tumor cells is scored 3+.

In more details, as above described, said appropriate scale is a scale of 0 to 3 wherein no membranous reactivity of tumor cells is scored 0; faint perceptible membranous reactivity in more than 10% of tumor cells is scored 1+; weak to moderate complete membranous reactivity in more than 10% of tumor cells is scored 2+; and strong complete reactivity in more than 10% of tumor cells is scored 3+.

In another embodiment of the invention, it is described a process of determining in vitro or ex vivo the status of a tumor from a subject, wherein said process comprises the steps of (a) scoring a tumor from a subject according to the simplified scale as above described; and (b) determining that the status of the tumor is [CXCR4(+)] with a score of 2+ or 3+; or (c) determining that the status of the tumor is [CXCR4(−)] with a score of 0 or 1+.

In a particular aspect of the invention, a tumor is [CXCR4(+)] with a score of 2+.

In a particular aspect of the invention, a tumor is [CXCR4(+)] with a score of 3+.

In another particular aspect of the invention, a tumor is [CXCR4(+)] with a score of 2+ or 3+.

Generally, the results of a test or assay according to the invention can be presented in any of a variety of formats. The results can be presented qualitatively. For example, the test report may indicate only whether or not a particular polypeptide was detected, perhaps also with an indication of the limits of detection. The results may be displayed as semi-quantitative. For example, various ranges may be defined, and the ranges may be assigned a score (e.g., 0 to 3+ or 0 to 8 depending on the used scale) that provides a certain degree of quantitative information. Such a score may reflect various factors, e.g., the number of cells in which CXCR4 is detected, the intensity of the signal (which may indicate the level of expression of CXCR4 or CXCR4-bearing cells), etc. The results may be displayed in a quantitative way, e.g., as a percentage of cells in which the polypeptide (CXCR4) is detected, as a protein concentration, etc.

As will be appreciated by one of ordinary skill in the art, the type of output provided by a test will vary depending upon the technical limitations of the test and the biological significance associated with detection of the polypeptide. For example, in the case of certain polypeptides a purely qualitative output (e.g., whether or not the polypeptide is detected at a certain detection level) provides significant information. In other cases a more quantitative output (e.g., a ratio of the level of expression of the polypeptide in the sample being tested versus the normal level) is necessary.

The invention also provides a method for determining whether an oncogenic disorder is susceptible to treatment with a anti-CXCR4 antibody, or a fragment or derivative thereof, wherein said process comprises the steps of:

(a) determining in vitro or ex vivo the CXCR4 status of a tumor of a subject according to the methods of the invention, and

(b) determining that, if the status is CXCR4(+), the oncogenic disorder is susceptible to treatment with an anti-CXCR4 antibody, or a fragment or derivative thereof.

In another aspect, the invention relates to a method of diagnosing pathological hyperproliferative oncogenic disorder or a susceptibility to a pathological condition associated with expression of CXCR4 in a subject, said method comprising the steps of:

(a) determining the presence or absence of CXCR4-carrying cells in a sample, and

(b) diagnosing a pathological condition or susceptibility to a pathological condition based on the presence or absence of said CXCR4 bearing cells.

In the methods of the invention, the detection of CXCR4-expressing cells or an increase in the levels of CXCR4 is generally indicative of a patient with or suspected of presenting a CXCR4-mediated disorder.

The present invention thus provides a method for predicting the risk of an individual to develop a cancer, said method comprising detecting the expression level of CXCR4 in a tissue sample, wherein a high level of CXCR4 expression is indicative of a high risk of developing a cancer.

It has been observed that CXCR4 expression is significantly associated with progressed tumor stages in several types of cancers (Schimanski et al., J Clin Oncol, ASCO Annual Meeting Proceedings Part I., 24(18S): 14018, 2006; Lee et al., Int J Oncol., 34(2):473-480, 2009; Pagano, Tesi di dottorato, Università degli Studi di Napoli Federico II, 2008). Thus the invention also relates to a method for evaluating tumor aggressiveness. “Tumor aggressiveness” as used herein refers to a tumor quickly growing and tending to spread rapidly.

In one embodiment, the said method comprises the step of:

(a) determining the level of CXCR4 expressed by cells in a tumor sample, and

(b) determining the level of CXCR4 expressed in an equivalent tissue sample taken from the same individual at a later time,

(c) determining the ratio between the expression level obtained in step (a) and the ratio obtained in step (b)

wherein the ratio of CXCR4 expression in the tumor sample over time provides information on the risks of cancer progression.

In a preferred embodiment, a ratio of the level obtained in step (a) to the level obtained in step (b) greater than 1 indicates aggressiveness. In another embodiment, a ratio inferior or equal to 1 indicates non aggressiveness.

Another aspect of the invention is the monitoring of CXCR4 expression in response to the administration of a CXCR4-targeted therapy. Such a monitoring can be very useful when the said therapy triggers the downregulation and/or the degradation of CXCR4.

In particular, monitoring CXCR4 expression on the cell surface could be a critical tool for evaluating the efficacy of the treatment during clinical trials and “personalized” therapies.

The application thus provides methods for determining the appropriate therapeutic regimen for a subject.

An increase or a decrease in the level of CXCR4 is indicative of the evolution of a cancer associated with CXCR4. Thus, by measuring an increase in the number of cells expressing CXCR4 or changes in the concentration of CXCR4 present in various tissues or cells, it is possible to determine whether a particular therapeutic regimen aimed at ameliorating a malignancy associated with CXCR4 is effective.

Therefore, the present invention is also directed to a method for determining the efficacy of a therapeutic regimen designed to alleviate an oncogenic disorder associated with CXCR4 in a subject suffering from said disorder, the said method comprising the steps of:

(a) determining a first expression level of CXCR4 in a first biological sample, said biological sample corresponding to first time point of the said treatment;

(b) determining a second expression level of CXCR4 in a second biological sample, said second biological sample corresponding to a second, later time point of the said treatment;

(c) calculating the ratio of the said first expression level obtained in step (a) to the said second expression level obtained in step (b); and

(d) determining that the efficacy of said therapeutic regimen is high when the ratio of step (c) is greater than 1; or

(e) determining that the efficacy of said therapeutic regimen is low when the ratio of step (c) is inferior or equal to 1.

In a preferred embodiment, the said therapeutic regime designed to alleviate an oncogenic disorder associated with CXCR4 in a subject suffering from said disorder includes the administration of a CXCR4 inhibitor to the said subject.

Another preferred embodiment of the invention relates to a method for selecting a cancer patient predicted to benefit, or not, from the administration of a therapeutic amount of a CXCR4 inhibitor, the said method comprising the steps of:

(a) determining the expression level of CXCR4 according to the methods of the invention;

(b) comparing the expression level obtained in step (a) with a reference expression level; and

(c) selecting the patient as being predicted to benefit from the administration of a therapeutic amount of a CXCR4 inhibitor, if the ratio of the expression level obtained in (a) to the reference expression level is greater than 1; or

(d) selecting the patient as being not predicted to benefit from the administration of a therapeutic amount of a CXCR4 inhibitor, if the ratio of the expression level obtained in (a) to the reference expression level is equal to or smaller than 1.

In the sense of the present specification, the expression “CXCR4 inhibitor” is intended to encompass any compound or molecule capable of binding to CXCR4 and inhibiting the binding of the ligand. As a non limitative example, CXCR4 inhibitors include AMD3100 and AMD3465. Other CXCR4 inhibitors that may be used include but are not limited to CTCE-0214; CTCE-9908; CP-1221 (linear peptides, cyclic peptides, natural amino-acids, unnatural amino acids, and peptidomimetic compounds); T140 and analogs; 4F-benzoyl-TN24003; KRH-1120; KRH-1636; KRH-2731; polyphemusin analogue; ALX40-4C. Still other CXCR4 inhibitors are described in WO 01/85196; WO 99/50461; WO 01/94420; and WO 03/090512, each of which is incorporated herein by reference.

In a preferred embodiment, the CXCR4 inhibitor consists of the monoclonal antibody 515H7.

In the most preferred embodiment, the said CXCR4 inhibitor is the monoclonal antibody 515H7 (WO2010/037831).

It is also an object of the invention to provide an in vivo method of imaging an oncogenic disorder associated with expression of CXCR4 as monomer and/or homodimer. Such a method is useful for localizing in vivo the tumor, as well as monitoring its invasiveness. Likewise, the method is useful for monitoring the progression and/or the response to treatment in patients previously diagnosed with a monomeric/homodimeric CXCR-mediated cancer.

In one embodiment, the invention relates to a method for detecting the location of a CXCR4-expressing tumor in a subject, said method comprising the steps of:

a) administering the antibody I-3859, or an antigen-binding fragment or derivative thereof, to the subject; and

b) detecting binding of said antibody,

wherein said binding indicates the presence of the tumor.

In another embodiment, the invention relates to a method for detecting the location of a CXCR-expressing tumor in a subject, said method comprising the steps of:

(a) administering the antibody I-3859, or an antigen-binding fragment or derivative thereof, to the subject; and

(b) detecting the binding of said antibody,

wherein said binding indicates the location of the tumor.

As for the detection of the presence of an expressing tumor, many techniques known by the man skilled in the art can be used. Nevertheless, preferred means are IHC and FACS.

In another aspect, the invention provides an in vivo imaging reagent, the said reagent comprising an antibody according to the invention, or an antigen-binding fragment or derivative thereof, the said antibody or a fragment or derivative thereof being preferably labeled, more preferably radiolabeled. The said reagent can be administered to a patient suffering from a CXCR4-mediated cancer in combination with a pharmaceutically effective carrier.

The present invention also contemplates the use of the said reagent in medical imaging of a patient suffering from a CXCR4-mediated cancer.

The method of the invention comprises the steps of:

(a) administering to the said patient an imaging-effective amount of an imaging reagent and

(b) detecting the said reagent.

In one embodiment, the method of the invention allows detection of the presence of a CXCR4-expressing tumor in the said patient. In another embodiment, the method of the invention allows the detection of the location of a CXCR4-expressing tumor in the said patient.

In a preferred embodiment, the imaging agent comprises a targeting moiety and an active moiety.

As used herein, the term “targeting moiety” refers to an agent that specifically recognizes and binds CXCR4 on the cell surface. In a particular embodiment, the targeting moiety is an antibody or a fragment or a derivative thereof which binds specifically to CXCR4. Specifically, the targeting moiety is an antibody or a fragment or derivative thereof as described above. Preferably, the targeting moiety is the I-3859 antibody.

An “active moiety” as used herein is an agent which permits in vivo detection of the said imaging reagent. The active moiety according to the invention includes in particular radio-elements such as Technetium-99m (99mTc), Copper-67 (Cu-67), Scandium-47 (Sc-47), Luthetium-77 (Lu-177) copper-64(Cu-64), Yttrium-86 (Y-86) or Iodine-124 (I-124).

The imaging agent is administered in an amount effective for diagnostic use in a mammal such as a human and the localization and accumulation of the imaging agent is then detected. The localization and accumulation of the imaging agent may be detected by radionucleide imaging, radioscintigraphy, nuclear magnetic resonance imaging, computed tomography, positron emission tomography, computerized axial tomography, X-ray or magnetic resonance imaging method, fluorescence detection, and chemiluminescent detection.

With regards to the development of targeted antitumor therapy, the diagnosis with immunohistological techniques gives in situ information on the receptor expression level, e.g. as regards the size and/or the location of the tumor. The diagnosis thus enables to select patients susceptible to be treated following the expression level of receptors needed for such a treatment.

More particularly, the CXCR4 expression level is measured preferentially by Fluorescence Activated Cell Sorting (FACS) or imunohistochemistry (IHC).

FACS analysis is extensively used in immunology and hematology to assess the presence of different cellular populations within a heterogeneous cell suspension. The number of monoclonal antibodies available for FACS analysis is very large, and they are coupled to different fluorochromes, allowing an easy multiple antigen staining. Immunophenotype is an essential parameter in the diagnosis of hematological malignancies. FACS analysis is used in the analysis of bone marrow, peripheral blood samples and tissues biopsies with suspected hematological malignancies (Martinez A. Cytometry Part B (Clinical Cytometry) 2003 56B 8-15). For example, Fiedler W et al. (Fiedler W. Blood 2003 102 2763-2767) reported the use of FACS analysis to screen AML patients for c-kit expression before treatment with SU5416, a small molecule inhibiting the phosphorylation of the VEGF receptors 1 and 2, c-kit, the SCF receptor and fms-like tyrosine kinase-3 (FLT3).

A “biological sample” may be any sample that may be taken from a subject. Such a sample must allow for the determination of the expression levels of the biomarker of the invention. The nature of the sample will thus be dependent upon the nature of the tumor. Preferred biological samples for the determination of the said biomarkers expression level by detection of the activated Ala and/or Erk proteins include samples such as a blood sample, a plasma sample, or a lymph sample, if the cancer is a liquid tumor. By “liquid tumor”, it is herein referred to tumors of the blood or bone marrow, i.e. hematologic malignancies such as leukemia and multiple myeloma. Preferably, the biological sample is a blood sample. Indeed, such a blood sample may be obtained by a completely harmless blood collection from the patient and thus allows for a non-invasive diagnosis of a CXCR4-inhbitor responding or non-responding phenotype.

A “biological sample” as used herein also includes a solid cancer sample of the patient to be tested, when the cancer is a solid cancer. Such solid cancer sample allows the skilled person to perform any type of measurement of the level of the biomarker of the invention. In some cases, the methods according to the invention may further comprise a preliminary step of taking a solid cancer sample from the patient. By a “solid cancer sample”, it is referred to a tumor tissue sample. Even in a cancerous patient, the tissue which is the site of the tumor still comprises non tumor healthy tissue. The “cancer sample” should thus be limited to tumor tissue taken from the patient. Said “cancer sample” may be a biopsy sample or a sample taken from a surgical resection therapy.

According to one aspect, the sample from the patient is a cancer cell or a cancer tissue.

This sample may be taken and if necessary prepared according to methods known to a person skilled in the art.

The cancer cell or cancer tissue in the present invention is not particularly limited.

As used herein, the term “cancer” refers to or describes the physiological condition in mammals that is typically characterized by unregulated cell proliferation. The terms “cancer” and “cancerous” as used herein are meant to encompass all stages of the disease. Thus, a “cancer” as used herein may include both benign and malignant tumors. Examples of cancer include but are not limited to, carcinoma, lymphoma, blastoma, sarcoma, and leukemia or lymphoid malignancies. More specifically, a cancer according to the present invention is selected from the group comprising squamous cell cancer (e.g., epithelial squamous cell cancer), lung cancer including small-cell lung cancer, non-small cell lung cancer, adenocarcinoma of the lung and squamous carcinoma of the lung, cancer of the peritoneum, hepatocellular cancer, gastric or stomach cancer including gastrointestinal cancer and gastrointestinal stromal cancer, pancreatic cancer, glioblastoma, cervical cancer, ovarian cancer, liver cancer, bladder cancer, cancer of the urinary tract, hepatoma, breast cancer, colon cancer, rectal cancer, colorectal cancer, endometrial or uterine carcinoma, salivary gland carcinoma, kidney or renal cancer, prostate cancer, vulval cancer, thyroid cancer, hepatic carcinoma, anal carcinoma, penile carcinoma, melanoma, superficial spreading melanoma, lentigo maligna melanoma, acral lentiginous melanomas, nodular melanomas, multiple myeloma and B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AID S-related lymphoma; and Waldenstrom's Macroglobulinemia); chronic lymphocytic leukemia (CLL); acute lymphoblastic leukemia (ALL); hairy cell leukemia; chronic myeloblastic leukemia (CML); Acute Myeloblastic Leukemia (AML); and post-transplant lymphoproliferative disorder (PTLD), as well as abnormal vascular proliferation associated with phakomatoses, edema (such as that associated with brain tumors), Meigs' syndrome, brain, as well as head and neck cancer, and associated metastases.

In a preferred embodiment, said cancer is selected among prostate cancer, osteosarcoma, lung cancer, breast cancer, endometrial cancer, leukemia, lymphoma, multiple myeloma, ovarian cancer, pancreatic cancer and colon cancer. In a more preferred embodiment, said cancer comprises lymphoma cell, leukemia cell or multiple myeloma cell.

The expression level of CXCR4 is advantageously compared or measured in relation to levels in a control cell or sample also referred to as a “reference level” or “reference expression level”. “Reference level”, “reference expression level”, “control level” and “control” are used interchangeably in the specification. A “control level” means a separate baseline level measured in a comparable control cell, which is generally disease or cancer free. The said control cell may be from the same individual, since, even in a cancerous patient, the tissue which is the site of the tumor still comprises non tumor healthy tissue . . . it may also originate from another individual who is normal or does not present with the same disease from which the diseased or test sample is obtained. Within the context of the present invention, the term “reference level” refers to a “control level” of expression of CXCR4 used to evaluate a test level of expression of CXCR4 in a cancer cell-containing sample of a patient. For example, when the level of CXCR4 in the biological sample of a patient is higher than the reference level of CXCR4, the cells will be considered to have a high level of expression, or overexpression, of CXCR4. The reference level can be determined by a plurality of methods. Expression levels may thus define CXCR4 bearing cells or alternatively the level of expression of CXCR4 independent of the number of cells expressing CXCR4. Thus the reference level for each patient can be prescribed by a reference ratio of CXCR4, wherein the reference ratio can be determined by any of the methods for determining the reference levels described herein.

For example, the control may be a predetermined value, which can take a variety of forms. It can be a single cut-off value, such as a median or mean. The “reference level” can be a single number, equally applicable to every patient individually, or the reference level can vary, according to specific subpopulations of patients. Thus, for example, older men might have a different reference level than younger men for the same cancer, and women might have a different reference level than men for the same cancer. Alternatively, the “reference level” can be determined by measuring the level of expression of CXCR4 in non-oncogenic cancer cells from the same tissue as the tissue of the neoplastic cells to be tested. As well, the “reference level” might be a certain ratio of CXCR4 in the neoplastic cells of a patient relative to the CXCR4 levels in non-tumor cells within the same patient. The “reference level” can also be a level of CXCR4 of in vitro cultured cells, which can be manipulated to simulate tumor cells, or can be manipulated in any other manner which yields expression levels which accurately determine the reference level. On the other hand, the “reference level” can be established based upon comparative groups, such as in groups not having elevated CXCR4 levels and groups having elevated CXCR4 levels. Another example of comparative groups would be groups having a particular disease, condition or symptoms and groups without the disease. The predetermined value can be arranged, for example, where a tested population is divided equally (or unequally) into groups, such as a low-risk group, a medium-risk group and a high-risk group.

The reference level can also be determined by comparison of the level of CXCR4 in populations of patients having the same cancer. This can be accomplished, for example, by histogram analysis, in which an entire cohort of patients are graphically presented, wherein a first axis represents the level of CXCR4, and a second axis represents the number of patients in the cohort whose tumor cells express CXCR4 at a given level. Two or more separate groups of patients can be determined by identification of subsets populations of the cohort which have the same or similar levels of CXCR4. Determination of the reference level can then be made based on a level which best distinguishes these separate groups. A reference level also can represent the levels of two or more markers, one of which is CXCR4. Two or more markers can be represented, for example, by a ratio of values for levels of each marker.

Likewise, an apparently healthy population will have a different ‘normal’ range than will have a population which is known to have a condition associated with expression of CXCR4. Accordingly, the predetermined value selected may take into account the category in which an individual falls. Appropriate ranges and categories can be selected with no more than routine experimentation by those of ordinary skill in the art. By “elevated” “increased” it is meant high relative to a selected control. Typically the control will be based on apparently healthy normal individuals in an appropriate age bracket.

It will also be understood that the controls according to the invention may be, in addition to predetermined values, samples of materials tested in parallel with the experimental materials. Examples include tissue or cells obtained at the same time from the same subject, for example, parts of a single biopsy, or parts of a single cell sample from the subject.

In another embodiment, the invention relates to a pharmaceutical composition for in vivo imaging of an oncogenic disorder associated with expression of CXCR4 comprising the above monoclonal antibody or a fragment thereof which is labeled and which binds CXCR4 in vivo; and a pharmaceutically acceptable carrier.

In another aspect of the invention, a kit useful for such diagnosing or prognosing process is provided, said kit comprising the antibody of the invention, or a fragment or derivative thereof

A kit, useful for detecting the presence and/or location of a CXCR4-expressing tumor, can include at least one of:

a) an antibody, or an antigen-binding fragment or derivative thereof, comprising i) a heavy chain comprising the following three CDRs, respectively CDR-H1 having the sequence SEQ ID No. 1, CDR-H2 having the sequence SEQ ID No. 2 and CDR-H3 having the sequence SEQ ID No. 3; and ii) a light chain comprising the following three CDRs, respectively CDR-L1 having the sequence SEQ ID No. 4, CDR-L2 having the sequence SEQ ID No. 5 and CDR-L3 having the sequence SEQ ID No. 6;

b) an antibody with a heavy chain comprising the following three CDRs, respectively CDR-H1 having the sequence SEQ ID No. 1, CDR-H2 having the sequence SEQ ID No. 2 and CDR-H3 having the sequence SEQ ID No. 3; and a light-chain variable domain comprising the sequence SEQ ID No. 8;

c) an antibody with a heavy chain variable domain comprising the sequence SEQ ID No. 7; and a light chain comprising the following three CDRs, respectively CDR-L1 having the sequence SEQ ID No. 4, CDR-L2 having the sequence SEQ ID No. 5 and CDR-L3 having the sequence SEQ ID No. 6;

d) an antibody with a heavy chain variable domain comprising the sequence SEQ ID No. 7; and a light-chain variable domain comprising the sequence SEQ ID No. 8.

Packaged materials comprising a combination of reagents in predetermined amounts with instructions for performing the diagnostic assay, e.g. kits, are also within the scope of the invention. The kit contains the antibodies for detection and quantification of CXCR4 in vitro, e.g. in an ELISA. The antibody of the present invention can be provided in a kit for detection and quantification of CXCR4 in vitro, e.g. in an ELISA. Where the antibody is labeled with an enzyme, the kit will include substrates and cofactors required by the enzyme (e.g., a substrate precursor which provides the detectable chromophore or fluorophore). In addition, other additives may be included such as stabilizers, buffers (e.g., a block buffer or lysis buffer) and the like. Such a kit may comprise a receptacle being compartmentalized to receive one or more containers such as vials, tubes and the like, such containers holding separate elements of the invention. For example, one container may contain a first antibody bound to an insoluble or partly soluble carrier. A second container may contain soluble, detectably-labeled second antibody, in lyophilized form or in solution. The receptacle may also contain a third container holding a detectably labeled third antibody in lyophilized form or in solution. A kit of this nature can be used in the sandwich assay of the invention. The label or package insert may provide a description of the composition as well as instructions for the intended in vitro or diagnostic use.

The relative amounts of the various reagents may be varied widely to provide for concentrations in solution of the reagents which substantially optimize the sensitivity of the assay. Particularly, the reagents may be provided as dry powders, usually lyophilized, including excipients which on dissolution will provide a reagent solution having the appropriate concentration.

In yet a further aspect of the invention, monoclonal antibodies or binding fragments thereof as detailed herein are provided labeled with a detectable moiety, such that they may be packaged and used, for example, in kits, to diagnose or identify cells having the aforementioned antigen. Non-limiting examples of such labels include fluorophores such as fluorescein isothiocyanate; chromophores, radionuclides, biotine or enzymes. Such labeled antibodies or binding fragments may be used for the histological localization of the antigen, ELISA, cell sorting, as well as other immunological techniques for detecting or quantifying CXCR4, and cells bearing this antigen, for example.

Kits are also provided that are useful as a positive control for purification or immunoprecipitation of CXCR4 from cells. For isolation and purification of CXCR4, the kit can contain the antibodies described herein or antigen-binding fragments thereof coupled to beads (e.g., sepharose beads). Kits can be provided which contain the antibodies for detection and quantitation of CXCR4 in vitro, e.g. in an ELISA. The kit comprises a container and a label or package insert on or associated with the container. The container holds a composition comprising at least the antibody I-3859, or an antigen-binding fragment or derivative thereof, of the invention. Additional containers may be included that contain, e.g., diluents and buffers, control antibodies. The label or package insert may provide a description of the composition as well as instructions for the intended in vitro or diagnostic use.

More particularly, the invention concerns a kit for the determination of the CXCR4 status of a tumor by the methods of the invention. In a preferred embodiment, as it will be described in the example, the invention relates to a kit for the determination of the CXCR4 status of a tumor by IHC and/or FACS methods.

In a particular embodiment, the invention consists in a kit comprising at least the antibody I-3859, or an antigen-binding fragment or derivative thereof, as above described, said antibody being labeled.

In a preferred embodiment, the kit ac cording to the invention further comprises a reagent useful for detecting the extent of binding between the said antibody I-3859 and CXCR4.

In another preferred embodiment, the kit of the invention useful for determining in vitro or ex vivo the expression level of CXCR4 in a CXCR4-expressing tumor, further comprises a reagent useful for quantifying the level of binding between the said labeled antibody and CXCR4.

In still another embodiment, the kit according to the invention further comprises: i) a reagent useful for detecting the extent of binding between the said labeled antibody and CXCR4; and ii) positive and negative control samples useful for the scoring the CXCR4 expression level.

Said kit can further comprise a polyclonal antibody specific to murine antibodies, preferably said polyclonal antibody specific to murine antibodies is labeled.

According to a particular embodiment of the invention, the kit for selecting in vitro a cancer patient who is predicted to benefit or not benefit from therapeutic administration of a CXCR4 inhibitor can comprise: i) a reagent useful for detecting the extent of binding between the said antibody and CXCR4; ii) control level that has been correlated with sensitivity to a CXCR4 inhibitor and/or iii) control level that has been correlated with resistance to a CXCR4 inhibitor.

The invention also relates to an in vivo or ex vivo diagnostic reagent composed of the antibody according to the invention, or an antigen-binding fragment or derivative thereof, preferably labeled, notably radiolabeled, and its use in medical imaging, notably for the detection of cancer related to the cellular expression or overexpression of CXCR4.

Other characteristics and advantages of the invention appear in the continuation of the description with the examples and the figures whose legends are represented below.

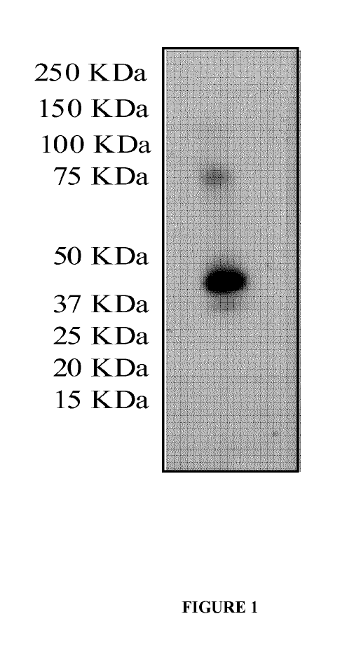

FIG. 1 shows that I-3859 Mab immunoprecipitates both CXCR4 monomers and dimers.

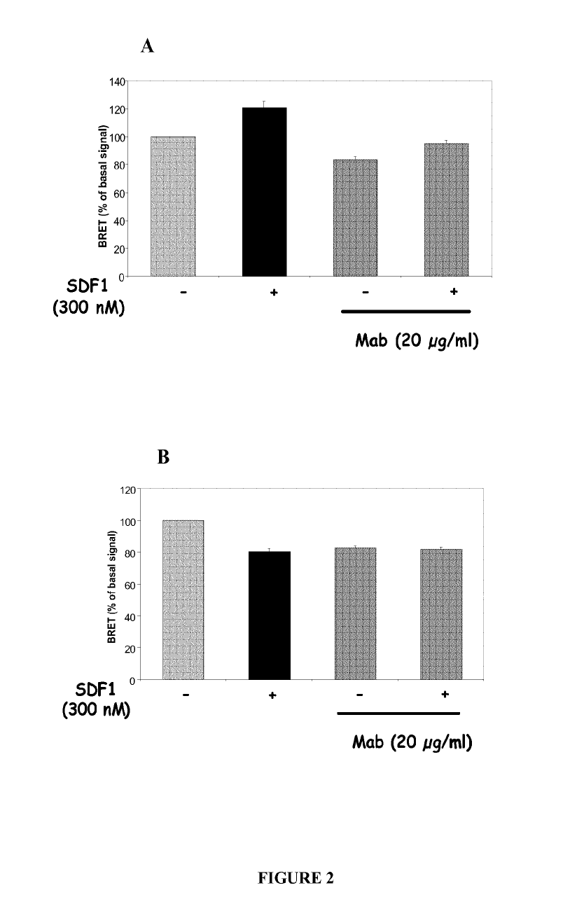

FIGS. 2A and 2B show that I-3859 Mab modulates both CXCR4 homodimers (A) and CXCR4/CXCR2 heterodimers (B).

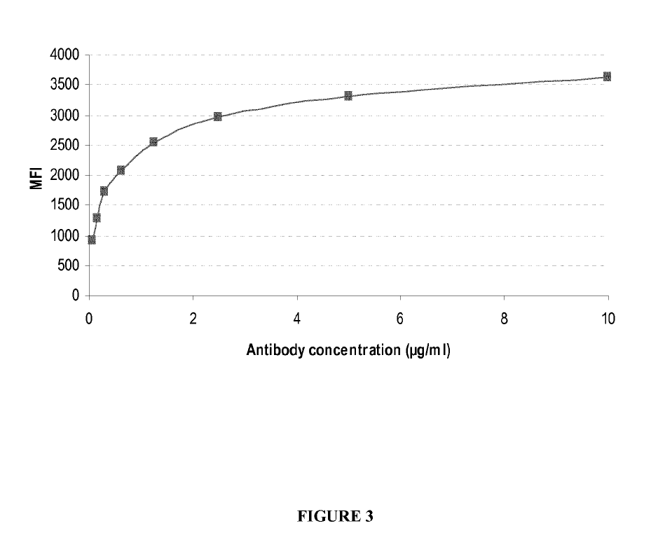

FIG. 3 shows that I-3859 Mab recognizes CXCR4 at the cell membrane by FACS analysis.

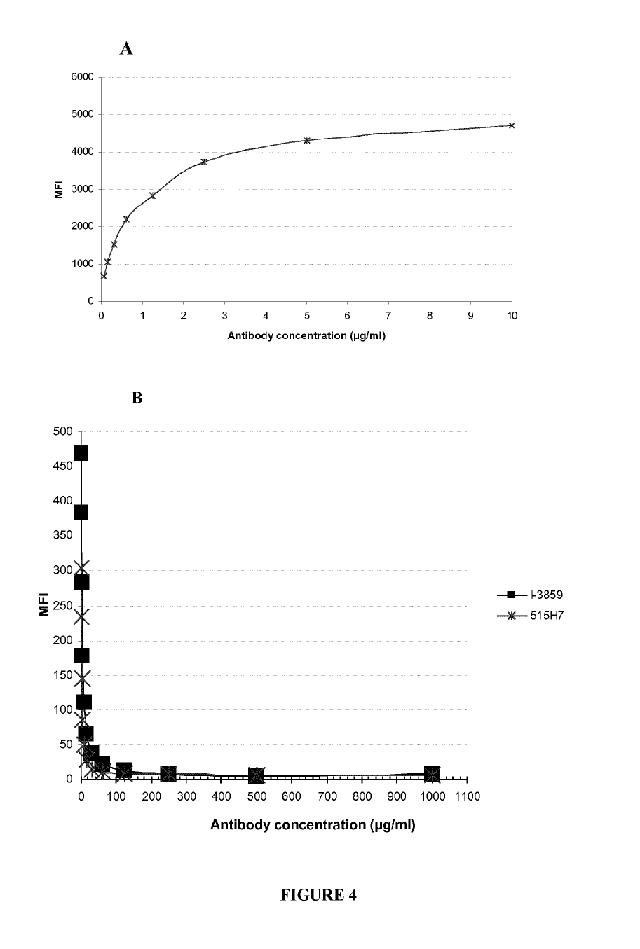

FIGS. 4A and 4B show that I-3859 Mab enters in competition with the anti-CXCR4 515H7 therapeutic Mab for binding to CXCR4 at cell membrane, by FACS analysis.

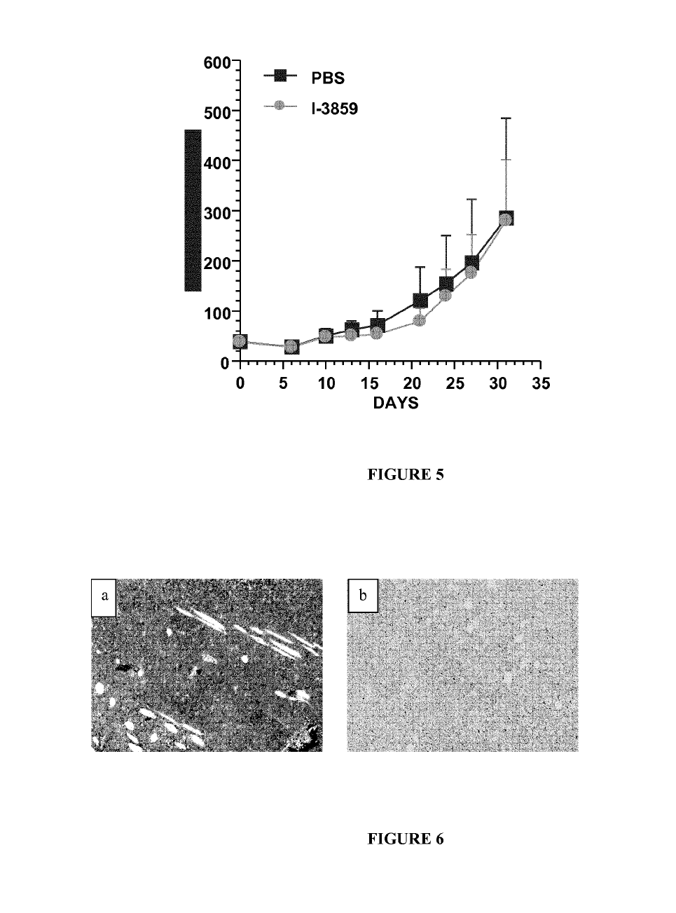

FIG. 5 shows that I-3859 Mab has no effect on MDA-MB-231 xenograft tumor growth model in Athymic nude mice.

FIG. 6 illustrates IHC staining using a) I-3859 and b) mIgG1 on RAMOS xenograft tumor.

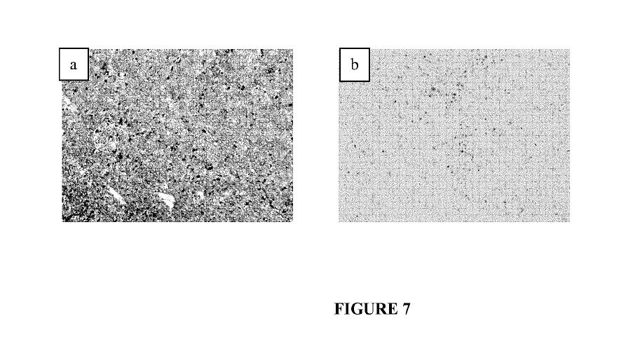

FIG. 7 illustrates IHC staining using a) I-3859 and b) mIgG1 on KARPAS299 xenograft tumors.

### EXAMPLE 1

### Anti-CXCR4 I-3859 Monoclonal Antibody (Mab) Generation

To generate monoclonal antibodies to CXCR4, Balb/c mice were immunized with recombinant NIH3T3-CXCR4 cells and/or peptides corresponding to CXCR4 extracellular N-term and loops. The mice 6-16 weeks of age upon the first immunization, were immunized once with the antigen in complete Freund's adjuvant subcutaneously (s.c.) followed by 2 to 6 immunizations with antigen in incomplete Freund's adjuvant s.c. The immune response was monitored by retroorbital bleeds. The serum was screened by ELISA (as described bellow) and mice with the higher titers of anti-CXCR4 antibodies were used for fusions. Mice were boost intravenously with antigen two days before sacrifice and removal of the spleen.

ELISA

To select the mice producing anti-CXCR4 antibodies, sera from immunized mice was tested by ELISA. Briefly, microtiter plates were coated with purified [1-41] N-terminal peptide conjugated to BSA at 5 μg equivalent peptide/mL, 100 μL/well incubated at 4° C. overnight, then blocked with 250 μL/well of 0.5% gelatine in PBS. Dilutions of plasma from CXCR4-immunized mice were added to each well and incubated 2 hours at 37° C. The plates were washed with PBS and then incubated with a goat anti-mouse IgG antibody conjugated to HRP (Jackson Laboratories) for 1 hour at 37° C. After washing, plates were developed with TMB substrate, the reaction was stopped 5 min later by addition of 100 μL/well 1M H₂SO₄. Mice that developed the highest titers of anti-CXCR4 antibodies were used for antibody generation.

Generation of Hybridomas Producing Mabs to CXCR4

The mouse splenocytes, isolated from a Balb/c mice that developed the highest titers of anti-CXCR4 antibodies were fused with PEG to a mouse myeloma cell line Sp2/O. Cells were plated at approximately 1×10⁵/well in microtiter plates followed by two weeks incubation in selective medium containing ultra culture medium+2 mM L-glutamine+1 mM sodium pyruvate+1×HAT. Wells were then screened by ELISA for anti-CXCR4 monoclonal IgG antibodies. The antibody secreting hybridomas were then subcloned at least twice by limiting dilution, cultured in vitro to generate antibody for further analysis.

### EXAMPLE 2

### I-3859 Mab Immunoprecipitates Both CXCR4 Monomers and Dimers

NIH3T3-CXCR4 cell pellets were washed with 20 mM TrisHCl, pH 8.5 containing 100 mM (NH4)₂SO₄ and then suspended in lysis buffer (20 mM TrisHCl, pH 8.5 containing 100 mM (NH4)₂SO₄, 10% glycerol, 1% CHAPSO and 10 μL/mL protease inhibitor cocktail). Cells were disrupted with Potter Elvehjem homogenizer. The solubilized membranes were collected by centrifugation at 105000 g at +4° C. for 1 h, then incubated overnight at +4° C. with I3859 Mab-coupled Sepharose 4B beads and mixture was poured into a glass column and washed with lysis buffer. The proteins captured by I3859 Mab were eluted and analyzed by western blot using an anti-CXCR4 Mab as primary antibody. Interesting fractions were pooled, concentrated and used for both WB analysis and preparative SDS-PAGE resolution (4-12% Bis-Tris gel).

After silver staining, the bands of interest were excised from the gel and submitted to in gel digestion using an automated protein digestion system, MassPREP station (Waters, Milford, Mass., USA). The gel spots were washed twice with 50 μL of 25 mM NH₄HCO₃ (Sigma, Steinheim, Germany) and 50 μL of acetonitrile (Carlo Erba Reactifs-SDS, Val de Reuil, France). The cysteine residues were reduced at 60° C. for 1 hour by 50 μL of 10 mM DTT prepared in 25 mM NH₄HCO₃ and alkylated at room temperature for 20 minutes by 50 μL of 55 mM iodoacetamide (Sigma) prepared in 25 mM NH₄HCO₃. After dehydration of the gel spots with acetonitrile, the proteins were digested overnight in gel by adding 10 μL of 12.5 ng/μl modified porcine trypsin (Promega, Madison, Wis., USA) in 25 mM NH₄HCO₃ at room temperature. The generated peptides were extracted with 35 μL of 60% acetonitrile containing 5% formic acid (Riedel-de Haën, Seelze, Denmark) followed by removing acetonitrile excess and were subjected to nano-LC-MS/MS. Mass data collected during nanoLC-MS/MS analysis were processed and converted into *.mgf files to be submitted to the MASCOT™ search engine. Searches were performed with a tolerance on measurements of 0.25 Da in MS and MS/MS modes.

FIG. 1 shows western blot analysis of eluted concentrated fractions after immunoprecipitation using I-3859 Mab-coupled Sepharose beads. Two bands at 43 and 75 kDa apparent molecular weights were stained by an anti-CXCR4 Mab used as primary antiboby.

Eluted concentrated fraction after immunoprecipitation using I-3859 Mab-coupled Sepharose beads was also resolved by SDS-PAGE and visualized by silver staining. The bands at 43 and 75 KDa were excised from gel, digested with trypsin and analyzed by LC-MS/MS as described above. The collected peak lists were submitted to Mascot for peptide sequence database search. CXCR4 was identified in both bands:

Five CXCR4 peptides were identified in the 75-kDa band via the MASCOT™ search engine: 31-38 peptide EENANFNK, contained in N-terminal CXCR4; 71-77 peptide SMTDKYR, contained in intra-cellular loop 1; 311-322 peptide TSAQHALTSVSR, 312-322 peptide SAQHALTSVSR, 313-322 peptide AQHALTSVSR contained in C-terminal.

Nine CXCR4 peptides were identified in the 43-kDa band via the MASCOT™ search engine: 27-30 peptide PCFR, 31-38 peptide EENANFNK, contained in N-terminal; 71-77 peptide SMTDKYR, contained in intra-cellular loop 1; 135-143 peptide YLAIVHATN and 135-146 peptide YLAIVHATNSQR, contained in intra-cellular loop 2; 311-319 peptide TSAQHALTS, 311-322 peptide TSAQHALTSVSR, 312-322 peptide SAQHALTSVSR, 313-322 peptide AQHALTSVSR contained in C-terminal.

The results obtained in this study clearly show that the I-3859 Mab is capable of immunoprecipitating CXCR4. The I-3859 Mab recognizes CXCR4 both as monomers and dimers.

### EXAMPLE 3

### The I-3859 Mab Modulates Both CXCR4 Homodimers and CXCR4/CXCR2 Heterodimers by BRET Analysis

This functional assay allows evaluating the conformational changes induced upon SDF-1 and/or I-3859 Mab binding to CXCR4 receptor at the level of CXCR4 homodimer and CXCR2/CXCR4 heterodimer formation.

Expression vectors for each of the investigated interaction partners were constructed as fusion proteins with the corresponding dye (Renilla reniformis luciferase, Rluc and Yellow fluorescent protein, YFP) by applying conventional molecular biology techniques. Two days prior performing BRET experiments, HEK293 cells were transiently transfected with expression vectors coding for the corresponding BRET partners: [CXCR4/Rluc+CXCR4/YFP] to study CXCR4 homodimerization and [CXCR4-Rluc+CXCR2-YFP] to study CXCR4 and CXCR2 heterodimerization. The day after, cells were distributed in poly-lysine pre-coated white 96 MW plates in complete culture medium [DMEM supplemented with 10% FBS]. Cells were first cultivated at 37° C. with CO₂ 5% in order to allow cell attachment to the plate. Cells were then starved with 200 μl DMEM/well overnight. Immediately prior to the BRET experiment, DMEM was removed and cells were quickly washed with PBS. Cells were then incubated in PBS in the presence or absence of antibody, 15 min at 37° C. prior to the addition of coelenterazine H 5 μM with or without SDF-1 in a final volume of 50 μl. After incubation for 5 minutes at 37° C. and further incubation for 20 min at room temperature, light-emission acquisition at 485 nm and 530 nm was initiated using the Mithras LB940 multilabel reader (Berthold) (1 s/wavelength/well repeated 15 times at room temperature).

Calculation of the BRET ratio was performed as previously described (Angers et al., 2000): [(emission_{530 nm})−(emission_{485 nm})×Cf]/(emisSion_{485 nm}), where Cf=(emission_{530 nm})/(emission_{485 nm}) for cells expressing the Rluc fusion protein alone under the same experimental conditions. Simplifying this equation shows that BRET ratio corresponds to the ratio 530/485 nm obtained when the two BRET partners are present, corrected by the ratio 530/485 nm obtained under the same experimental conditions, when only the partner fused to Rluc is present in the assay. For sake of readability, results are expressed in percentage of the basal signal.

Addition of SDF1 (300 nM) triggered an increase of the BRET signal, resulting from the spatial proximity of the adaptor and acceptor proteins fused to the CXCR4 receptor, by about 20%. This increase is likely to indicate either CXCR4/CXCR4 homodimers formation or conformational changes of pre-existing dimers (FIG. 2A). The I-3859 Mab was capable of modulating SDF-1-induced conformational changes for CXCR4 homodimers (69% inhibition of SDF-1-induced BRET increase). The I-3859 Mab was also capable of modulating by itself CXCR4/CXCR4 spatial proximity, indicating an influence of the I-3859 Mab on CXCR4/CXCR4 homodimer conformation (FIG. 2A).

The BRET signal resulting from the spatial proximity of CXCR4 and CXCR2 receptors was decreased by about 20% in response to SDF1 (300 nM). This result suggests CXCR4/CXCR2 heterodimers formation or conformational changes of pre-existing dimers (FIG. 2B). The I-3859 Mab was capable of modulating SDF-1-induced conformational changes for CXCR2/CXCR4 heterodimer with a percentage of inhibition of SDF-1-induced BRET decrease of about 100% and was also capable of modulating by itself CXCR4/CXCR2 spatial proximity, indicating an influence of the I-3859 Mab on CXCR4/CXCR2 heterodimers conformation (FIG. 2B).

### EXAMPLE 4

### The I-3859 Mab Recognizes CXCR4 Present at the Cell Surface by FACS Analysis

In this experiment, specific binding of the I-3859 Mab to human CXCR4 was examined by FACS analysis.

The NIH3T3, NIH3T3-hCXCR4 transfected cells, MDA-MB-231, Hela, and U937 cancer cell lines were incubated with the I-3859 monoclonal antibody. The cells were then washed with 1% BSA/PBS/0.01% NaN3. Next, Alexa-labeled secondary antibodies were added to the cells and were allowed to incubate at 4° C. for 20 min. The cells were then washed again two times. Following the second wash, FACS analysis was performed. Results of these binding studies are provided in FIG. 3; they show that the anti-CXCR4 Mab I-3859 binds to human CXCR4-NIH3T3 transfected cell line [Mean Fluorescence Intensity (MFI)] whereas there was no recognition of the parent NIH3T3 cells (not shown). This Mab was also capable of recognizing human cancer cell lines, for examples MDA-MB-231 breast cancer cells (MFI at a concentration of 10 μg/ml=59), U937 promyelocytic cancer cells (MFI at a concentration of 10 μg/ml=246) and Hela cervix cancer cells (MFI at a concentration of 10 μg/ml=633), indicating that these cell lines naturally overexpress CXCR4.

### EXAMPLE 5

### I-3859 Mab Enters in Competition with the Anti-CXCR4 515H7 Therapeutic Mab for Binding to CXCR4 at Cell Membrane by FACS Analysis

In this experiment, competition of binding to human CXCR4 of the I-3859 and 515H7 Mabs was examined by FACS analysis.

NIH3T3-hCXCR4 transfected cells were incubated with biotinylated 515H7 Mab (5 μg/ml) [which recognized NIH3T3-CXCR4 cells (FIG. 4A)] and either the I-3859 Mab or 515H7 Mab (0-1 mg/mL) for 1 hour at 4° C. The cells were then washed with 1% BSA/PBS/0.01% NaN3. Next, labeled-streptavidin was added to the cells and was allowed to incubate at 4° C. for 20 min. The cells were then washed again two times. Following the second wash, FACS analysis was performed. Results of these binding studies are provided in FIG. 4B. They show [Mean Fluorescence Intensity (MFI)] that the anti-CXCR4 Mab I-3859 competes with the anti-CXCR4 515H7 therapeutic Mab for binding to human CXCR4-NIH3T3 transfected cells. As expected, the non-labeled 515H7 Mab also inhibited the binding of the biotinylated 515H7 Mab to CXCR4.

### EXAMPLE 6

### I-3859 Mab Activity Evaluation in MDA-MB-231 Xenograft Tumor Growth Model in Athymic Nude Mice

The goal of this experiment was to evaluate the ability of the anti-CXCR4 Mab I-3859 to inhibit the growth of a MDB-MB-231 xenograft in Athymic nude mice.

MDA-MB-231 cells from ECACC were routinely cultured in DMEM medium (Invitrogen Corporation, Scotland, UK), 10% FCS (Sigma, St Louis Md., USA). Cells were split 48 hours before engraftment so that they were in exponential phase of growth. Ten million MDA-MB-231 cells were engrafted in PBS to 7 weeks old Athymic nude mice (Harlan, France). Five days after implantation, tumors were measurable (34 mm³<V³<40 mm³) and animals were divided into groups of 12 mice with comparable tumor size. Mice were treated i.p. with a 2 mg/mouse loading dose of the I-3859 Mab. Then, mice were injected twice a week at 1 mg/dose/mouse of the I-3859 Mab. A PBS group was introduced as a control group in this experiment. Tumor volume was measured twice a week and calculated by the formula: π/6× length×width×height. Statistical analyses were performed at each measure using a Mann-Whitney test.

In this experiment, no mortality was observed during treatment. Compared to PBS group, there was no significant inhibition of tumor growth at D31 for the I-3859 Mab (1 mg/dose). The average tumor volume after 4 weeks of treatment was not reduced by the I-3859 Mab versus PBS (FIG. 5).

### EXAMPLE 7

### Immunohistochemical Studies (IHC)

Sections were deparaffinized, rehydrated, and placed at 37° C. for 10 minutes in pre-warm protease 1 buffer (Ventana Medical system) for heat-induced epitope retrieval. After 3 washes in Tris Buffer Saline-0.05% tween 20 (TBS-T) (Dako S3006) the endogenous peroxidase activity was blocked using Peroxidase Blocking Reagent (Dako K4007) for five minutes. Sections were washed with TBS-T and incubated in blocking reagent (UltraV block-TA-125UB-LabVision) for 5 minutes before incubation with the I-3859 (15 μg/ml, clone I-3859, Pierre Fabre) or mouse IgG1/kappa (15 μg/ml, X0931, Dako) as an isotype control overnight at 4° C. Sections were washed with TBS-T and incubated with SignalStain Boost IHC detection Reagent (HRP, M) for 30 minutes at room temperature. Diaminobenzidine was used for development of a brown reaction product (Dako K3468). The slides were immersed in hematoxylin for 4 minutes to counterstain (Dako S3309) and washed in PBS before being mounted in Faramount mounting medium plus coverslipe. In this immunohistochemistry procedure, the brown reaction product correlates to positive staining of the cell membrane and lack of brown reaction product correlates to negative staining and no visualization of the cell membrane.

The I-3859 Mab, differentially stains the cell membrane of various tumor types. FIGS. 6 and 7 illustrated staining performed in 2 xenograft models in which is described an anti-tumoral activity with the therapeutic anti-CXCR-4 hz515H7 antibody: RAMOS and KARPAS299. As shown in FIGS. 6 and 7, the expression detected is lower in KARPAS299 (FIG. 7) xenograft than in RAMOS (FIG. 6). This data well correlate with the study of the CXCR-4 expression by flow cytometry. Indeed, RAMOS cells express about 5 levels more of CXCR-4 than KARPAS299 one (Antibody Binding Capacity: 200 000 for RAMOS and 40 000 for KARPAS299). Membranous staining is weaker in KARPAS299 (FIG. 7), whereas, membranous staining is significantly higher in RAMOS (FIG. 6).

1. An antibody, or an antigen-binding fragment or derivative thereof, for use in detecting the presence and/or location of a CXCR4-expressing tumor, said antibody comprising i) a heavy chain comprising the following three CDRs, respectively CDR-H1 having the sequence SEQ ID No. 1, CDR-H2 having the sequence SEQ ID No. 2 and CDR-H3 having the sequence SEQ ID No. 3; and ii) a light chain comprising the following three CDRs, respectively CDR-L1 having the sequence SEQ ID No. 4, CDR-L2 having the sequence SEQ ID No. 5 and CDR-L3 having the sequence SEQ ID No. 6.

2. The antibody according to claim 1, or an antigen binding fragment or derivative thereof, characterized in that it is selected among:
a) an antibody with a heavy chain comprising the following three CDRs, respectively CDR-H1 having the sequence SEQ ID No. 1, CDR-H2 having the sequence SEQ ID No. 2 and CDR-H3 having the sequence SEQ ID No. 3; and a light-chain variable domain comprising the sequence SEQ ID No. 8;
b) an antibody with a heavy chain variable domain comprising the sequence SEQ ID No. 7; and a light chain comprising the following three CDRs, respectively CDR-L1 having the sequence SEQ ID No. 4, CDR-L2 having the sequence SEQ ID No. 5 and CDR-L3 having the sequence SEQ ID No. 6; or
c) an antibody with a heavy chain variable domain comprising the sequence SEQ ID No. 7; and a light-chain variable domain comprising the sequence SEQ ID No. 8.

3. The antibody, or an antigen-binding fragment or derivative thereof, of anyone of claim 1 or 2, for use in in vitro or ex vivo diagnosing or prognosing an oncogenic disorder associated with expression of CXCR4.

4. The antibody, or an antigen-binding fragment or derivative thereof, of anyone of claims 1 to 3, wherein the said antibody has no in vivo anti-tumoral activity.

5. A method for detecting in vitro or ex vivo the presence and/or the location of a CXCR4-expressing tumor in a subject, said method comprising the steps of:
(a) contacting a biological sample from the subject with an antibody, or an antigen-binding fragment or derivative thereof, capable of binding specifically to CXCR4; and
(b) detecting the binding of the said antibody, or antigen-binding fragment or derivative thereof, with the said biological sample,
wherein the said antibody, or antigen-binding fragment or derivative thereof, comprises i) a heavy chain comprising the following three CDRs, respectively CDR-H1 having the sequence SEQ ID No. 1, CDR-H2 having the sequence SEQ ID No. 2 and CDR-H3 having the sequence SEQ ID No. 3; and ii) a light chain comprising the following three CDRs, respectively CDR-L1 having the sequence SEQ ID No. 4, CDR-L2 having the sequence SEQ ID No. 5 and CDR-L3 having the sequence SEQ ID No. 6.

6. A method for detecting in vitro or ex vivo the percentage of cells expressing CXCR4 in a subject, said method comprising the steps of:
(a) contacting a biological sample from the subject with an antibody, or an antigen-binding fragment or derivative thereof, capable of binding specifically to CXCR4; and
(b) quantifying the percentage of cells expressing CXCR4 in the biological sample,
characterized in that the said antibody, or antigen-binding fragment or derivative thereof, comprises i) a heavy chain comprising the following three CDRs, respectively CDR-H1 having the sequence SEQ ID No. 1, CDR-H2 having the sequence SEQ ID No. 2 and CDR-H3 having the sequence SEQ ID No. 3; and ii) a light chain comprising the following three CDRs, respectively CDR-L1 having the sequence SEQ ID No. 4, CDR-L2 having the sequence SEQ ID No. 5 and CDR-L3 having the sequence SEQ ID No. 6.

7. A method for determining in vitro or ex vivo the expression level of CXCR4 in a CXCR4-expressing tumor from a subject, said method comprising the steps of:
(a) contacting a biological sample from the subject with an antibody, or an antigen-binding fragment or derivative thereof, capable of binding specifically to CXCR4; and
(b) quantifying the level of binding of the said antibody, or antigen-binding fragment or derivative thereof, to CXCR4 in the said biological sample,
characterized in that the said antibody, or antigen-binding fragment or derivative thereof, comprises i) a heavy chain comprising the following three CDRs, respectively CDR-H1 having the sequence SEQ ID No. 1, CDR-H2 having the sequence SEQ ID No. 2 and CDR-H3 having the sequence SEQ ID No. 3; and ii) a light chain comprising the following three CDRs, respectively CDR-L1 having the sequence SEQ ID No. 4, CDR-L2 having the sequence SEQ ID No. 5 and CDR-L3 having the sequence SEQ ID No. 6.

8. The method of claim 7, wherein the level of binding of the said antibody, or antigen-binding fragment or derivative thereof, to CXCR4 is preferentially measured by Fluorescence Activated Cell Sorting (FACS) or immunohistochemistry (IHC).

9. A method of in vitro or ex vivo diagnosing or prognosing a CXCR4-expressing tumor, said method comprising the steps of:
(a) determining the expression level of CXCR4 according to claim 7 or 8, and
(b) comparing the expression level of step (a) with a reference expression level of CXCR4 from normal tissue or CXCR4-non expressing tissue.

10. A method for determining in vitro or ex vivo the scoring of a tumor of a subject, said method comprising the steps of:
(a) contacting a biological sample from the subject with an antibody, or an antigen-binding fragment or derivative thereof, capable of binding specifically to CXCR4;
(b) quantifying the level of binding of the said antibody, or antigen-binding fragment or derivative thereof, to CXCR4 in the said biological sample; and
(c) scoring the tumor by comparing the quantified level of binding of the said antibody, or antigen-binding fragment or derivative thereof, from the subject to an appropriate scale,
characterized in that the said antibody, or antigen-binding fragment or derivative thereof, comprises i) a heavy chain comprising the following three CDRs, respectively CDR-H1 having the sequence SEQ ID No. 1, CDR-H2 having the sequence SEQ ID No. 2 and CDR-H3 having the sequence SEQ ID No. 3; and ii) a light chain comprising the following three CDRs, respectively CDR-L1 having the sequence SEQ ID No. 4, CDR-L2 having the sequence SEQ ID No. 5 and CDR-L3 having the sequence SEQ ID No. 6.

11. The method of claim 10, wherein said the appropriate scale is based on two parameters which are the intensity of the staining and the percentage of positive cells.

12. The method of anyone claim 10 or 11, wherein the said appropriate scale is a scale of 0 to 8 wherein no reactivity is scored 0, and a strong reactivity in a proportion of 67-100% proportion reactive is scored 8.

13. A method for determining in vitro or ex vivo the status of a tumor from a subject, said method comprising the steps of:
(a) scoring a tumor from a subject according to one of claim 10, 11 or 12; and
(b) determining that the status of the tumor is [CXCR4(+)] with a score of 3 to 8; or
(c) determining that the status of the tumor is [CXCR4(−)] with a score of 0 to 2.

14. The method of anyone of claim 10 or 11, wherein said appropriate scale is a scale of 0 to 3⁺ wherein no membranous reactivity of tumor cells is scored 0 and strong complete reactivity in more than 10% of tumor cells is scored 3⁺.

15. A method for determining in vitro or ex vivo the status of a tumor from a subject, said method comprising the steps of:
(a) scoring a tumor from a subject according to one of claim 10, 11 or 14; and
(b) determining that the status of the tumor is [CXCR4(+)] with a score of 2⁺ or 3⁺; or
(c) determining that the status of the tumor is [CXCR4(−)] with a score of 0 or 1⁺.

16. A method for determining whether an oncogenic disorder is susceptible to treatment with a anti-CXCR4 antibody, or a fragment or derivative thereof, said method comprising the steps of:
(a) determining in vitro or ex vivo the CXCR4 status of a tumor of a subject according to claim 13 or 15, and
(b) determining that, if the status is CXCR4(+), the oncogenic disorder is susceptible to treatment with an anti-CXCR4 antibody, or a fragment or derivative thereof.

17. A method for selecting a cancer patient predicted to benefit or not from the administration of a therapeutic amount of a CXCR4 inhibitor, said method comprising the steps of:
(a) determining the expression level of CXCR4 according to the method of claim 7 or 8;
(b) comparing the expression level of the previous step a) with a reference expression level; and
(c) selecting the patient as being predicted to benefit from therapeutic administration of a CXCR4 inhibitor, if the ratio of the expression level obtained in (a) to the reference expression level is greater than 1; or
(d) selecting the patient as being not predicted to benefit from therapeutic administration of a CXCR4 inhibitor, if the ratio of the expression level obtained in (a) to the reference expression level is inferior or equal to 1.

18. A method for determining in vitro or ex vivo the efficacy of a therapeutic regimen designed to alleviate an oncogenic disorder associated with CXCR4 in a subject suffering from said disorder, said method comprising the steps of:
(a) determining a first expression level of CXCR4 according to claim 7 or 8 in a first biological sample, said first biological sample corresponding to first time point of the said treatment;
(b) determining a second expression level of CXCR4 according to claim 7 or 8 in a second biological sample, said second biological sample corresponding to a second, later time point of the said treatment;
(c) calculating the ratio of the said first expression level obtained in step (a) to the said second expression level obtained in step (b); and
(d) determining that the efficacy of said therapeutic regime is high when the ratio of step (c) is greater than 1; or
(e) determining that the efficacy of said therapeutic regime is low when the ratio of step (c) is inferior or equal to second expression level is statistically similar to or.

19. The method of claim 18, wherein the said therapeutic regime designed to alleviate an oncogenic disorder associated with CXCR4 in a subject suffering from said disorder includes the administration of a CXCR4 inhibitor to the said subject.

20. A kit for detecting the presence and/or location of a CXCR4-expressing tumor, the said kit including at least one of:
a) an antibody, or an antigen-binding fragment or derivative thereof, comprising i) a heavy chain comprising the following three CDRs, respectively CDR-H1 having the sequence SEQ ID No. 1, CDR-H2 having the sequence SEQ ID No. 2 and CDR-H3 having the sequence SEQ ID No. 3; and ii) a light chain comprising the following three CDRs, respectively CDR-L1 having the sequence SEQ ID No. 4, CDR-L2 having the sequence SEQ ID No. 5 and CDR-L3 having the sequence SEQ ID No. 6;
b) an antibody with a heavy chain comprising the following three CDRs, respectively CDR-H1 having the sequence SEQ ID No. 1, CDR-H2 having the sequence SEQ ID No. 2 and CDR-H3 having the sequence SEQ ID No. 3; and a light-chain variable domain comprising the sequence SEQ ID No. 8;
c) an antibody with a heavy chain variable domain comprising the sequence SEQ ID No. 7; and a light chain comprising the following three CDRs, respectively CDR-L1 having the sequence SEQ ID No. 4, CDR-L2 having the sequence SEQ ID No. 5 and CDR-L3 having the sequence SEQ ID No. 6;
d) an antibody with a heavy chain variable domain comprising the sequence SEQ ID No. 7; and a light-chain variable domain comprising the sequence SEQ ID No. 8.

21. The kit of claim 20, wherein the said antibody is labeled.

22. The kit of anyone of claim 20 or 21, further comprising a reagent for detecting the extent of binding between the said antibody and CXCR4.

23. The kit of anyone of claim 20 or 21, further comprising a reagent for quantifying the level of binding between the said antibody and CXCR4.

24. The kit of anyone of claim 20 or 21, further comprising:
i) a reagent for detecting the extent of binding between the said antibody and CXCR4; and
ii) positive and negative control samples useful for the scoring the CXCR4 expression level.

25. The kit of claim 24, further comprising a polyclonal antibody specific to murine antibodies, said polyclonal antibody being preferably labeled.

26. The kit of anyone of claim 20 or 21, further comprising:
i) a reagent useful for detecting the extent of binding between the said antibody and CXCR4;
ii) control level that has been correlated with sensitivity to a CXCR4 inhibitor and/or
iii) control level that has been correlated with resistance to a CXCR4 inhibitor.
